(19) United States
(12) Patent Application Publication
(10) Pub. No.: US 2024/0290443 A1
(43) Pub. Date: Aug. 29, 2024

(54) METHODS TO CHARACTERIZE FUNCTIONAL AND DYSFUNCTIONAL ACUTE INFLAMMATORY RESPONSES TO PATHOLOGIC PROCESSES

(71) Applicant: The General Hospital Corporation, Boston, MA (US)

(72) Inventors: John M. Higgins, Cambridge, MA (US); Brody H. Foy, Boston, MA (US); Aaron Dominic Aguirre, Boston, MA (US); Jonathan C. Carlson, Boston, MA (US)

(21) Appl. No.: 18/571,154

(22) PCT Filed: Jun. 16, 2022
(86) PCT No.: PCT/US2022/072988

§ 371 Date: Dec. 15, 2023

Related U.S. Application Data
(60) Provisional application No. 63/211,513, filed on Jun. 16, 2021.

(51) Int. Cl. G16H 10/40; G16H 10/60; G16H 50/20

(52) U.S. Cl. CPC G16H 10/40; G16H 10/60; G16H 50/20

(57) ABSTRACT

Systems and methods for generating a trajectory model characterizing a disease state and using the trajectory model in determining a recommended treatment. In one aspect, a method includes obtaining patient data, in which the patient data includes clinical laboratory results of a patient with a disease state; processing the patient data such that the clinical laboratory results are normalized by patient-specific or cohort-specific baseline levels of the clinical laboratory results; and applying a trajectory model to the processed patient data to identify a likelihood of an adverse outcome of the patient, in which the trajectory model characterizes the disease state by a recovery trajectory in a phase-plane of one or more variables.

### CLAIM OF PRIORITY

This application claims the benefit of U.S. Provisional Application Ser. No. 63/211,513, filed on Jun. 16, 2021. The entire contents of the foregoing are incorporated herein by reference.

### FEDERALLY SPONSORED RESEARCH OR DEVELOPMENT

This invention was made with government support under grant number DP2DK098087 awarded by the National Institutes of Health. The government has certain rights in the invention.

### TECHNICAL FIELD

The disclosure relates to techniques for modeling human acute inflammatory recovery.

### BACKGROUND

Superscript numeric references in this disclosure generally refer to documents listed at the end of the detailed description.

Trauma, infection, ischemia, and other pathologic processes trigger an inflammatory response, a set of orchestrated cellular and tissue-level mechanisms that seek to prevent further injury and to repair existing damage. The acute phase of inflammation is typically induced by exogenous molecules from pathogens or endogenous molecules activated by tissue stress or damage. White blood cells (WBCs) residing in damaged tissue and platelets (PLTs) aggregating and activating at sites of vascular injury are key mediators of the downstream response. Homeostatic setpoints for blood cell populations are altered temporarily during an acute inflammatory event before returning to baseline during recovery. While the identities of many molecular inducers and cellular mechanisms have been established, response dynamics at the level of WBC and PLT effector populations are poorly understood. Leukocytosis (elevated WBC count) is a cardinal sign of acute inflammation, but simple elevation in WBC count is highly non-specific, and the rates of change and resolution in WBC and PLT associated with favorable acute inflammatory responses are not well-defined. Patient responses appear to vary dramatically with no clearly defined signs of good prognosis. This fragmented understanding of inflammatory responses at the cellular population level often limits clinical practice to binarized assessments of acute phase reactants and heuristic interpretation of blood counts.

### SUMMARY

There exists a need for understanding the dynamics of inflammatory recovery by using longitudinal clinical laboratory measurements. Inflammation is the physiologic reaction to cellular and tissue damage caused by trauma, ischemia, infection, and other pathologic processes. While elevation of white blood cell count (WBC) and altered levels of other acute phase reactants are cardinal signs of inflammation, the dynamics of these changes and their resolution are not well understood. The techniques described here present a novel approach to model the dynamics of an inflammatory response. The approach described herein enables predicting relative risk of patients in a computationally efficient manner: a trajectory model, which streamlines this process, is used to determine how well a patient is recovering from pathologic processes, and based on the determination, the patient can be scored based on the risk level.

By analyzing multivariate temporal relationships in clinical laboratory studies from patients responding to multiple types of infection, ischemia, and trauma associated with surgery, examples of the model revealed a pattern involving co-regulation of WBC and platelets (PLTs) populations. Specifically, uncomplicated recoveries were characterized by exponential decay from a maximum WBC followed by delayed linear growth of PLT. These examples of the model that explain this robust pattern of a universal recovery trajectory were also shown to be highly predictive for identifying high-risk patients, e.g., those at increased risk of adverse outcomes (e.g., death) from the pathologic process triggering the inflammation (e.g., infection) or from the inflammatory process spiraling out of control, and provided a benchmark for healthy inflammatory recovery.

In general, in a first aspect, a method includes obtaining, by one or more processors, patient data. The patient data includes clinical laboratory results of a patient with a disease state. The method includes processing, by the one or more processors, the patient data such that the clinical laboratory results are normalized by patient-specific baseline levels or cohort-specific baseline levels of the clinical laboratory results. The cohort-specific baseline levels specify levels of the clinical laboratory results (e.g., reference ranges of WBC, PLT, and other measurements) before the inflammatory recovery starts. The normalization considers the relative risk of the patient, by comparing a given patient to other patients with similarity, where the other patients are identified by statistical analysis. The method includes applying, by the one or more processors, a trajectory model to the processed patient data to identify a likelihood of an adverse outcome of the patient. The trajectory model characterizes the disease state by a recovery trajectory in a phase-plane of one or more variables and is configured to compare the processed patient data to the recovery trajectory and to output, based on the comparing, the likelihood of adverse outcome of the patient.

In general, in a second aspect, combinable with the first aspect, the method further includes providing, based on the likelihood of adverse outcome of the patient, information indicative of a recommended treatment.

In general, in a third aspect, combinable with the second aspect, the recommended treatment includes a recommendation to continue a current treatment regimen.

In general, in a fourth aspect, combinable with the second aspect, the recommended treatment includes a recommendation to modify a current treatment regimen.

In general, in a fifth aspect, combinable with the first aspect, the clinical laboratory results are indicative of at least one of a blood count, a metabolic panel measurement, or a vital sign measurement.

In general, in a sixth aspect, combinable with the first aspect, the disease state includes an acute inflammatory response.

In general, in a seventh aspect, combinable with the sixth aspect, the acute inflammatory response is a response to a pathologic process or a surgical or procedural intervention.

In general, in an eighth aspect, combinable with the sixth aspect, the pathologic process is selected from the group consisting of: trauma, infection, ischemia, cancer, stroke, autoimmunity, and surgery.

In general, in a ninth aspect, combinable with the first aspect, the one or more variables include a white blood cell count.

In general, in a tenth aspect, combinable with the first aspect, the one or more variables include a white blood cell count and a platelet count.

In general, in an eleventh aspect, combinable with the first aspect, the one or more variables include a white blood cell count and a blood urea nitrogen level.

In general, in a twelfth aspect, combinable with the first aspect, the one or more variables include a white blood cell count and a red blood cell distribution width.

In general, in a thirteenth aspect, combinable with the first aspect, the one or more variables include a platelet count and a red blood cell distribution width.

In general, in a fourteenth aspect, combinable with the first aspect, the trajectory model is predictive of the adverse outcome.

In general, in a fifteenth aspect, combinable with the fourteenth aspect, the adverse outcome includes at least one outcome selected from the group consisting of: a complication and mortality.

In general, in a sixteenth aspect, combinable with the first aspect, the recovery trajectory includes an exponential decay of white blood cell count and a linear increase in platelet count.

In general, in a seventeenth aspect, combinable with the first aspect, comparing the processed patient data to the recovery trajectory includes identifying positions of the processed patient data relative to the recovery trajectory in the phase plane; and determining a degree to which the positions of the processed patient data deviate from the recovery trajectory at any given time point and over serial time points.

In general, in an eighteenth aspect, combinable with the seventeenth aspect, determining the degree to which the positions of the processed patient data deviate from the recovery trajectory includes computing a direction or angle between the positions of the processed patient data and the recovery trajectory in the phase-plane.

In general, in a nineteenth aspect, combinable with the first aspect, the method further includes identifying, for the patient, the patient-specific or the cohort-specific baseline levels of the clinical laboratory results. The baseline levels of the clinical laboratory results represent levels of the one or more variables before the patient is diagnosed with the disease state.

In general, in a twentieth aspect, combinable with the first aspect, the method further includes obtaining second patient data of a plurality of patients. The plurality of patients does not include the patient, and the plurality of patients and the patient share patient attributes. The method includes imputing, based on the second patient data, missing values in the clinical laboratory results of the patient.

In general, in a twenty-first aspect, combinable with the twentieth aspect, the patient attributes include medical histories and demographic information.

In general, in a twenty-second aspect, combinable with the twenty-first aspect, each of the medical histories include historical clinical laboratory results for a respective patient.

In general, in a twenty-third aspect, combinable with the twentieth aspect, the patient attributes are used to determine a baseline risk of the patient. The baseline risk is useful for predicting the one or more variables.

In general, in a twenty-fourth aspect, combinable with the first aspect, the trajectory model further outputs a likelihood of a full and healthy recovery of the patient.

In general, in a twenty-fifth aspect, combinable with the first aspect, the method further includes obtaining training patient data. The training patient data includes clinical laboratory results of a plurality of patients with one or more disease states. The method includes processing the training patient data such that the clinical laboratory results of the plurality of patients are normalized by the patient-specific or cohort-specific baseline levels; identifying the one or more variables to be used in the phase plane; and fitting the trajectory model, using the one or more variables, to the plurality of training patient data.

In general, in a twenty-sixth aspect, combinable with the twenty-fifth aspect, the clinical laboratory results include measurements of at least one parameter selected from the group consisting of: anion gap, blood-urea nitrogen, creatinine, hematocrit, glucose, platelet count, red cell distribution width, and white blood cell count.

In general, in a twenty-seventh aspect, combinable with the twenty-fifth aspect, identifying the one or more variables includes: identifying, by applying unsupervised clustering to the plurality of training patient data, high-dimensional clusters, wherein the clusters are associated with the one or more disease states; reducing dimensionality of the high-dimensional clusters; and identifying the one or more variables that are significantly associated with the one or more disease states.

In general, in a twenty-eighth aspect, combinable with the twenty-seventh aspect, the unsupervised clustering includes k-means clustering and hierarchical clustering.

In general, in a twenty-ninth aspect, combinable with the twenty-seventh aspect, identifying the one or more variables that are significantly associated with the one or more disease states includes: for a different set of the one or more variables: computing a significance of a generalized linear model predicting the adverse outcome using the one or more variables in the plurality of training patient data; and determining that the significance meets a threshold.

In general, in a thirtieth aspect, combinable with the twenty-fifth aspect, fitting the trajectory model includes: fitting an exponential decay of white blood cell count using the plurality of training data; and fitting a linear increase in platelet count using the plurality of training data.

In general, in a thirty-first aspect, combinable with any of the first through thirtieth aspects, a method includes obtaining training patient data. The training patient data includes clinical laboratory results of a plurality of patients with one or more disease states. The method includes processing the training patient data such that the clinical laboratory results of the plurality of patients are normalized by patient-specific or cohort-specific baseline levels. The method includes identifying one or more variables to be used in a phase plane. The method includes fitting a trajectory model, using the one or more variables, to the plurality of training patient data. The trajectory model characterizes the one or more disease states by a recovery trajectory in the phase plane.

In general, in a thirty-second aspect, combinable with any of the first through thirty-first aspects, a system includes one or more processors and one or more storage devices storing instructions that are operable, when executed by the one or more processors, to cause the one or more processors to perform operations including: obtaining, by one or more processors, patient data, wherein the patient data includes clinical laboratory results of a patient with a disease state; processing, by the one or more processors, the patient data such that the clinical laboratory results are normalized by patient-specific baseline levels or cohort-specific baseline levels of the clinical laboratory results; and applying, by the one or more processors, a trajectory model to the processed patient data to identify a likelihood of an adverse outcome of the patient, in which the trajectory model characterizes the disease state by a recovery trajectory in a phase-plane of one or more variables and is configured to: compare the processed patient data to the recovery trajectory; and output, based on the comparing, the likelihood of adverse outcome of the patient.

In general, in a thirty-third aspect, combinable with any of the first through thirty-second aspects, a non-transitory computer-readable medium, including software instructions, that when executed by a computer, cause the computer to execute operations including: obtaining, by the computer, patient data, wherein the patient data includes clinical laboratory results of a patient with a disease state; processing, by the computer, the patient data such that the clinical laboratory results are normalized by patient-specific baseline levels or cohort-specific baseline levels of the clinical laboratory results; and applying, by the computer, a trajectory model to the processed patient data to identify a likelihood of an adverse outcome of the patient, in which the trajectory model characterizes the disease state by a recovery trajectory in a phase-plane of one or more variables and is configured to: compare the processed patient data to the recovery trajectory; and output, based on the comparing, the likelihood of adverse outcome of the patient.

In general, in a thirty-fourth aspect, combinable with any of the first through thirty-third aspects, a system includes one or more processors and one or more storage devices storing instructions that are operable, when executed by the one or more processors, to cause the one or more processors to perform operations including: obtaining, by the one or more processors, training patient data, wherein the training patient data includes clinical laboratory results of a plurality of patients with one or more disease states; processing, by the one or more processors, the training patient data such that the clinical laboratory results of the plurality of patients are normalized by patient-specific or cohort-specific baseline levels; identifying, by the one or more processors, one or more variables to be used in a phase plane; and fitting a trajectory model, by the one or more processors and using the one or more variables, to the plurality of training patient data, in which the trajectory model characterizes the one or more disease states by a recovery trajectory in the phase plane.

In general, in a thirty-fifth aspect, combinable with any of the first through thirty-fourth aspects, a non-transitory computer-readable medium, including software instructions, that when executed by a computer, cause the computer to execute operations including: obtaining, by the computer, training patient data, wherein the training patient data includes clinical laboratory results of a plurality of patients with one or more disease states; processing, by the computer, the training patient data such that the clinical laboratory results of the plurality of patients are normalized by patient-specific or cohort-specific baseline levels; identifying, by the computer, one or more variables to be used in a phase plane; and fitting a trajectory model, by the computer and using the one or more variables, to the plurality of training patient data, in which the trajectory model characterizes the one or more disease states by a recovery trajectory in the phase plane.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a block diagram of an example system for determining risk of a patient based on a trajectory model.

FIG. 2A is a flowchart of example process for determining a recommended treatment of a patient based on a trajectory model

FIG. 2B is a flowchart of example process for training a trajectory model.

FIG. 3A shows a set of graphs of clinical laboratory results showing a recovery trajectory of the acute inflammatory response to non-emergency cardiac surgery.

FIG. 3B shows a chart summarizing association power of a set of paired parameters with patient outcome.

FIG. 3C shows a set of graphs indicative of recovery trajectories under various conditions.

FIG. 3D shows a graph indicative of a recovery trajectory and a set of charts showing deviation from the recovery trajectory is associated with adverse outcomes.

FIG. 4A shows a set of graphs indicative of recovery trajectories from various cohorts with different pathologic processes.

FIG. 4B shows a graph indicative of approximation of the recovery trajectory by a white blood cell count (WBC) and a platelet count (PLT).

FIG. 4C shows a graph of a universal recovery trajectory.

FIG. 4D shows a set of charts indicative of distributions of patients following the universal recovery trajectory.

FIG. 5A shows a set of graphs indicative of WBC model fits across all 12 cohorts.

FIG. 5B shows a set of graphs indicative of PLT model fits across all 12 cohorts.

FIG. 5C shows a set of graphs showing WBC-PLT dynamics of representative patients.

FIG. 6A shows a set of graphs indicative of longitudinal WBC-PLT trajectories.

FIG. 6B shows a heat map indicative of relative risk of the patient based on the patient position and direction percentiles.

FIG. 6C shows a heat map indicative of adverse outcomes likelihood based on the patient position and direction percentiles.

FIGS. 7A-G show that high-dimensional clusters of response to cardiac surgery defined from routine clinical laboratory tests.

FIGS. 8A-C show autocorrelations for test results throughout recovery from cardiac surgery.

FIG. 9 shows cross-correlations for test results throughout recovery from cardiac surgery.

FIG. 10A shows mean WBC-PLT trajectories for cardiac surgery patients stratified by gender.

FIG. 10B shows mean WBC-PLT trajectories for cardiac surgery patients stratified by surgery year.

FIG. 11 shows mean WBC-PLT trajectories for cardiac surgery patients with unfavorable outcomes.

FIGS. 12A-B show WBC-PLT trajectories for surgical cohorts stratified by length of hospital stay (LOS)

FIG. 13 shows mean fitted WBC and PLT parameters for 12 inflammatory cohorts.

FIG. 14 shows phase-plane trajectories for cardiac surgery patients with favourable outcomes using WBC sub-types from WBC differentials.

FIG. 15A shows mean WBC-PLT trajectories stratified by demographic and clinical factors for a myocardial infarction cohort.

FIG. 15B shows mean WBC-PLT trajectories stratified by demographic and clinical factors for a COVID-19 cohort.

FIG. 16 shows example patient trajectories and model fits for patients whose raw data did not fit well (25^{th} percentile or lower) to the trajectory model.

FIG. 17 shows example patient trajectories and model fits for patients whose raw data did fit well (75^{th} percentile or above) to the trajectory model.

FIG. 18 shows a heat map indicative of COVID-19 mortality risk stratified by distance percentiles.

FIG. 19 shows cardiac surgery recovery trajectories for alternate test result pairs.

FIG. 20 shows example cardiac surgery WBC-PLT trajectories stratified by average degree of deviation for the full cohort.

FIG. 21 shows a set of graphs indicative of WBT-PLT model fits for inflammatory cohorts other than a cardiac surgery cohort.

FIG. 22 shows a set of heat maps indicative of risk of death or long stay for various cohorts.

FIG. 23 shows reference position and direction percentiles for a cardiac surgery cohort.

FIG. 24 shows reference position and direction percentiles for a hip arthroplasty surgery cohort.

FIG. 25 shows reference position and direction percentiles for a colectomy cohort.

FIG. 26 shows reference position and direction percentiles for a myocardial infarction cohort.

FIG. 27 shows reference position and direction percentiles for a COVID-19 cohort.

FIG. 28 shows reference position and direction percentiles for a sepsis cohort.

FIG. 29 shows characteristics of various inflammatory cohorts.

FIG. 30 shows characteristics of high-dimensional clusters of cardiac surgery cohort.

FIGS. 31-33 show relative risk stratified by variables in cardiac surgery cohort.

FIG. 34 shows cohort sizes after applying exclusion criteria.

FIGS. 35-36 show mortality risk for cardiac surgery cohort stratified by direction percentiles.

FIG. 37 shows adverse outcomes likelihood stratified by distance percentiles for cardiac surgery cohort, hip arthroplasty cohort, colectomy cohort, myocardial infarction cohort, COVID-19 cohort, and sepsis cohort.

FIG. 38 shows measurement units and reference intervals for clinical laboratory results.

FIG. 39 shows an example user interface of using the trajectory model.

### DETAILED DESCRIPTION

It is hypothesized that inflammatory recoveries from diverse pathologic processes (e.g., trauma, infection, ischemia, cardiac surgery, stroke, and colectomy) would share a common pattern describing a healthy full recovery trajectory. The recovery trajectory is a trajectory of patient data, e.g., clinical laboratory results (such as white blood cell count (WBC) and platelet count (PLT)), that explains recovery of a patient with an inflammatory response. Described herein is the recovery trajectory, also referred to as a trajectory model. The recovery trajectory is built based on analysis of longitudinal dynamics of the patient data and can be used to predict relative risk of patients based on a likelihood of having adverse outcomes from pathologic processes. Upon identifying high-risk patients (those deviating from the recovery trajectory), systems and methods described herein generate a report indicative of recommended treatment to restore patients to the favorable recovery trajectory. Similarly, upon identifying patients closely adhering to a favorable recovery trajectory, systems and methods generate a report recommending continuation of current treatment.

The systems and methods of the present disclosure can have one or more of the following advantages. First, the trajectory model considers multivariate temporal relationships in clinical laboratory results. Unlike a single variable-based model (e.g., that relies on only WBC), the multivariate trajectory model can be more predictive. For example, in examples of the multivariate trajectory model, deviation from the trajectory model was associated with a 5 to 33 times increased relative risk of adverse outcomes across 12 inflammatory cohorts. Second, the trajectory model is built to be interpretable, as the trajectory model utilizes phase plane analysis, as opposed to principal component analysis. The interpretable trajectory model enables easily identifying high-risk patients and generating recommended treatment for those patients deviating from the favorable recovery trajectory. Third, the trajectory model generalizes across inflammatory recoveries from various pathologic processes and is robust to variation in patient age, sex, year of surgery, operation type, and baseline level of clinical laboratory measurements. Fourth, even when a given patient's data is limited, e.g., only a few clinical laboratory measurements are available, the trajectory model can be applied to the imputed patient data, where the patient data is imputed based on other patients who share similar attributes, e.g., medical history and demographics.

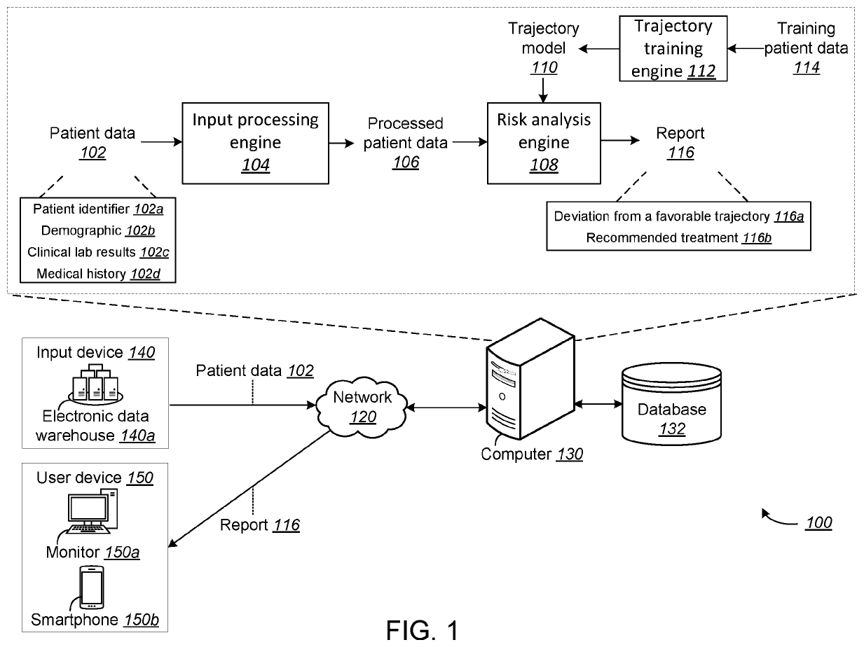

FIG. 1 is a block diagram of an example system 100 that obtains patient data 102 and generates a report 116 indicative of how much a patient deviates from a favorable trajectory and recommended treatment for the patient. The system 100 includes an input device 140, a network 120, and one or more computers 130 (e.g., one or more local or cloud-based processors). The computer 130 can include an input processing engine 104, a risk analysis engine 108, and a trajectory training engine 112. In some implementations, the computer 130 is a server.

The input device 140 is a device that is configured to obtain the patient data 102, a device that is configured to provide the patient data 102 to another device across the network 120, or any suitable combination thereof. For example, the input device 140 includes an electronic data warehouse 140a that stores clinical laboratory data of thousands of patients in a given hospital. In some implementations, the electronic data warehouse 140a can obtain the patient data 102, e.g., by accessing medical records of a patient, and transmit the patient data 102 to another device such as the computer 130 across the network 120. In some implementations, the electronic data warehouse 140a can obtain patient data 102 that can be accessed by one or more other input devices 140 such as computer (e.g., desktop, laptop, tablet, etc.), a smartphone, or a server. In such instances, the one or more other input devices can access the patient data 102 obtained by the electronic data warehouse 140a and transmit the obtained patient data 102 to the computer 130 via the network 120. The network 120 can include one or more of a wired Ethernet network, a wired optical network, a wireless WiFi network, a LAN, a WAN, a Bluetooth network, a cellular network, the Internet, or other suitable network, or any combination thereof. In some implementations, the electronic data warehouse 140a and the computer 130 are the same.

The computer 130 is configured to obtain patient data 102 from the input device 140. The patient data 102 includes a patient identifier 102a, demographic information 102b, clinical lab results 102c, and medical history 102d. In some implementations, the clinical lab results 102c include a blood count, a metabolic panel, or a vital sign measurement of a patient. In some implementations, the patient data 102 can be data received over the network 120. In some implementations, the computer 130 can store the patient data 102 in a database 132 and access the database 132 to retrieve the patient data 102. The database 132, such as a local database or a cloud-based database, can store the patient data 102 that are encrypted, including data for each of multiple patients, such as patient information (e.g., patient name, patient identifier, or other patient information such as demographic information), medical history (e.g., current pathologic processes, past visits to doctor's office, or other medical history), historic clinical lab results (e.g., blood count, metabolic panel, vital sign measurements), or other suitable data.

The input processing engine 104 is configured to receive the patient data 102 and generates processed patient data 106 for input to the risk analysis engine 108. The input processing engine 104 can perform one or more of standardization of a format of the patient data 102, imputation for missing data, data sampling (e.g., sampling lab results every 12 hours), or other data processing. For the standardization of a format of the patient data, the input processing engine 104 standardizes different units of the clinical lab results and different encodings of medical history and demographic information. For example, some physicians may use a full name of a pathologic process when recording medical history of a patient, while others may use an abbreviated name of the pathologic process. Standardizing the patient data 102 and training patient data 114 contribute to an accurate trajectory model. For the missing data imputation, the input processing engine 104 identifies multiple patients who share similar attributes to a patient with missing data, where the attributes include the demographic information 102b and the medical history 102d. For example, for a patient recovering from cardiac surgery, multiple other patients who are also recovering or already recovered from cardiac surgery can be used to impute clinical lab results of the patient. For the data sampling, the input processing engine 104 samples a number of lab results required for well-powered prediction of risk to reduce computational resources and time to be spent for downstream analysis (by the risk analysis engine 108). The processed patient data 106 outputted by the input processing engine 104 is provided as an input to the risk analysis engine 108.

The risk analysis engine 108 is configured to receive the processed patient data 106 and a trajectory model 110 and generate the report 116. The trajectory model 110 is generated from the trajectory training engine 112 trained on the training patient data 114. The training patient data 114 includes historical clinical lab results of multiple patients, who are different from a patient corresponding to the patient data 102. The trajectory training engine 112 performs phase plane analysis and models a recovery trajectory based on given variables, e.g., WBC and PLT counts, as a function of time. For example, the recovery trajectory shows exponential WBC decay and linear PLT growth (further described in examples below). The recovery trajectory outputted from the trajectory training engine 112 is referred to as the trajectory model 110. The risk analysis engine 108 compares recovery of a given patient to the trajectory model that characterizes a favorable inflammatory recovery and determines a degree to which the patient deviates from the favorable inflammatory recovery, generating deviation from a favorable trajectory 116a. In some implementations, the deviation 116a is quantified as a position (distance) and/or a direction percentile based on positions of the patient's clinical measurements on the favorable trajectory. For example, the deviation 116a specifies that the patient's direction percentile is 80%, and the patient's distance percentile is 50%. The favorable inflammatory recovery represents recovery from an acute inflammatory event with no or minimal adverse outcomes such as a complication and mortality and can be defined using clinical laboratory results of multiple patients, e.g., those who successfully recovered from various acute inflammatory events. For example, the risk analysis engine 108 computes position percentiles based on how well the patient's clinical lab measurements are fitted to the trajectory model 110 and compares the position percentiles against a threshold, where different threshold can be set for a different pathologic process. Responsive to the comparison, the risk analysis engine 108 determines a recommended treatment 116b as a part of the report 116.

The recommended treatment 116b can vary depending on the disease state the patient has. In some implementations, the recommended treatment 116b includes modifying a treatment regimen a patient has been receiving. For example, when the risk analysis engine 108 determines that the patient is at low-risk of complication as the patient's recovery aligns the recovery trajectory provided by the trajectory model 110, the risk analysis engine 108 generates the report 116 that specifies the patient to continue the current treatment regimen. On the other hand, when the risk analysis engine 108 determines that the patient is at high-risk as the patient's condition is predicted to deteriorate, the recommended treatment 116b modifies the current treatment regimen to more aggressive treatment regimen, e.g., by recommending more aggressive antibiotics. In this case, a clinician treating the high-risk patient can follow antibiotic escalation guidelines and improves the patient care by modifying the treatment regimen. As another example, the recommended treatment 116b for a patient with heart failure includes increasing diuresis or vasopressors. The computer 130 can generate rendering data that, when rendered by a device having a display such as a user device 150 (e.g., a computer having a monitor 150a, a mobile computing device such as a smart phone 150b, or another suitable user device), can cause the device to present on the display information indicative of the report 116, e.g., indicative of the deviation 116a from the favorable trajectory, indicative of the recommended treatment 116b, or indicative of both the deviation 116a and the recommended treatment 116b. Such rendering data can be transmitted, by the computer 130, to the user device 150 through the network 120 and processed by the user device 150 or associated processor to generate output data for display on the user device 150. In some implementations, the user device 150 can be coupled to the computer 130. In such instances, the rendered data can be processed by the computer 130, and cause the computer 130, on a user interface, to output data that include the report 116.

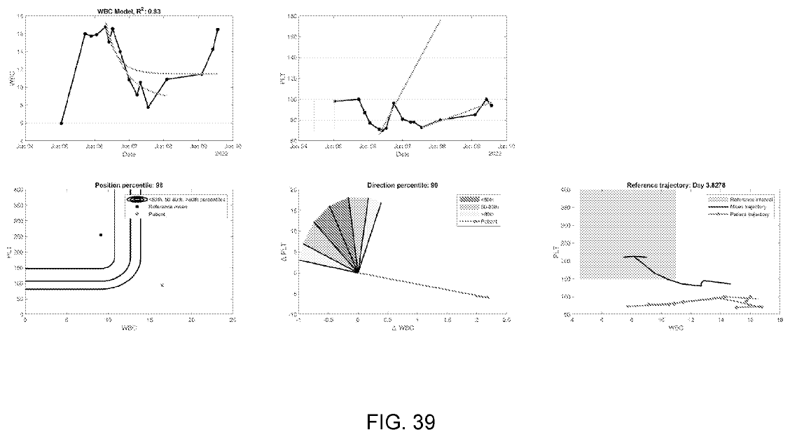

In some implementations, the user interface displays the processed patient data 106, e.g., overlaid on the trajectory model 110. For example, as shown in FIG. 39, WBC and PLT of a given patient were overlaid on the recovery trajectory. The user interface displayed the model fit correlation between the patient data and the trajectory mode. In some implementations, the user interface displays the deviation 116a, e.g., a position percentile and a direction percentile. For example, as shown in FIG. 39, the user interface displayed the position percentile of 98 and the direction percentile of 99.

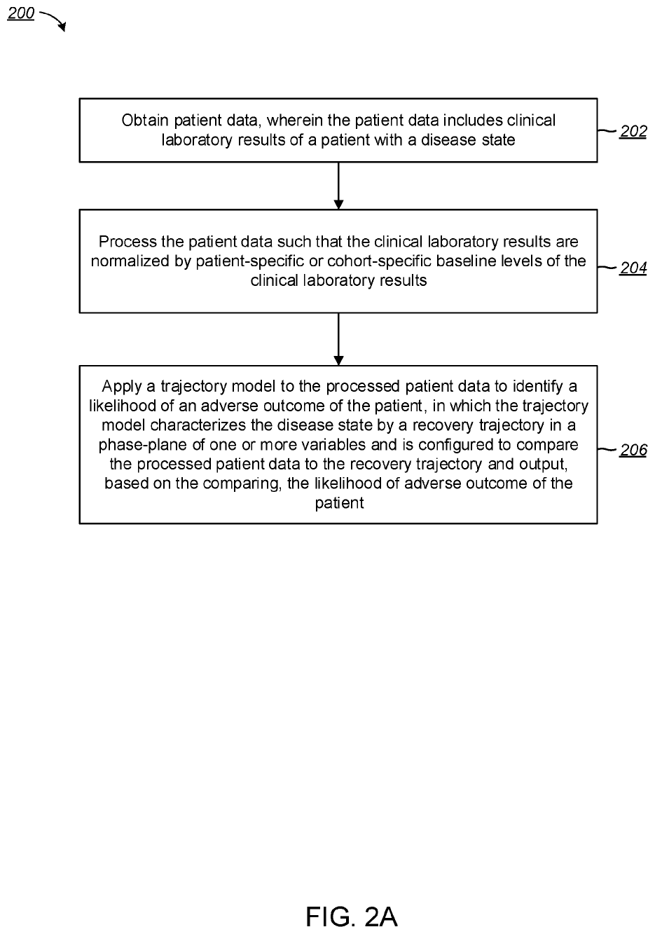

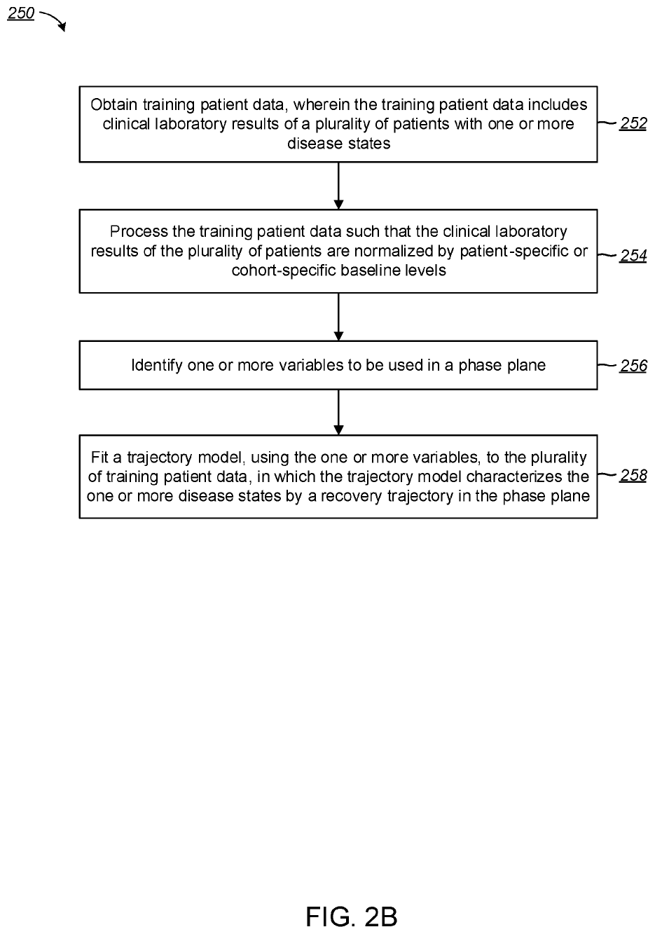

FIG. 2A is a flowchart of example process 200 for determining a recommended treatment of a patient based on a trajectory model. The process 200 will be described as being performed by a system of one or more computers programmed appropriately in accordance with this specification. For example, the system 100 of FIG. 1 can perform at least a portion of the example process. In some implementations, various steps of the process 200 can be run in parallel, in combination, in loops, or in any order.

The system obtains patient data (202). The patient data includes clinical laboratory results of a patient with a disease state. The clinical laboratory results are indicative of at least one of a blood count (e.g., white blood count), a metabolic panel (e.g., glucose level), or vital sign (e.g., blood pressure, pulse rate, body temperature) measurement. In some implementations, the disease state is an acute inflammatory response to a pathologic process or a surgical or procedural intervention. The pathologic process includes trauma (e.g., traffic accident, sexual violence), infection, ischemia, cancer (e.g., breast cancer, lung adenocarcinoma, and colorectal cancer), stroke, autoimmunity (e.g., rheumatoid arthritis), and cardiac surgery (e.g., aortic valve surgery, heart transplant).

The system processes the patient data such that the clinical laboratory results are normalized by patient-specific or cohort-specific baseline levels of the clinical laboratory results (204). The baseline levels of the clinical laboratory results represent the levels of a blood count or metabolic panel before the patient develops the disease state. For example, the clinical laboratory results of the patient, before the patient developed a lung adenocarcinoma, are used to determine the baseline levels (e.g., average fasting glucose level of 110 mg/dl). When the patient-specific baseline levels are not available, the cohort-specific baseline levels can be used, e.g., by using baseline levels of other patients similar to a given patient. The baseline levels can include summary statistics including a mean, a median, and a range computed on the historical clinical laboratory results. The system can also impute missing values in the patient data based on data from multiple other patients. For example, a white blood cell count of the patient can be imputed based on one or more other patients who have similar patient attributes, e.g., medical histories including historical clinical laboratory results and demographic information. The patient attributes are used to determine a baseline risk of the patient.

The system applies a trajectory model to the processed patient data to identify a likelihood of an adverse outcome of the patient (206). The trajectory model characterizes the disease state by a recovery trajectory in a phase-plane of one or more variables. In some implementations, the one or more variables include a white blood cell count. In some implementations, the variables include a white blood cell count and a platelet count. In some implementations, the variables include a white blood cell count and a blood urea nitrogen level. In some implementations, the variables include a white blood cell count and a red blood cell distribution width. In some implementations, the variables include a platelet count and a red blood cell distribution width.

The trajectory model is configured to compare the processed patient data to the recovery trajectory and output, based on the comparing, the likelihood of adverse outcome of the patient. The adverse outcome includes a complication (e.g., from the surgery) and mortality. In some implementations, the recovery trajectory is characterized by a decrease of the white blood cell count (e.g., an exponential decay) and an increase in the platelet count (e.g., a linear increase). In some implementations, the recovery trajectory is characterized by the white blood cell count (WBC) and the blood urea nitrogen level (BUN). In some implementations, the recovery trajectory is characterized by the WBC and the creatinine levels (CRE). In some implementations, the recovery trajectory is characterized by the WBC and the red cell distribution width (RDW). In some implementations, the recovery trajectory is characterized by the WBC and the anion gap (ANION). In some implementations, the recovery trajectory is characterized by the platelet count (PLT) and the BUN. In some implementations, the recovery trajectory is characterized by the RDW and the BUN. In some implementations, the recovery trajectory is characterized by the WBC and the hematocrit level (HCT). In some implementations, the recovery trajectory is characterized by the WBC and the glucose level (GLU). In some implementations, the recovery trajectory is characterized by the ANION and the BUN.

To compare the processed patient data to the recovery trajectory, the system identifies positions of the processed patient data relative to the recovery trajectory in the phase plane and determines a degree (e.g., distance and angle) to which the positions of the processed patient data deviate from the recovery trajectory.

In some implementations, the system further outputs a likelihood of a full and healthy recovery of the patient. The full and healthy recovery of the patient refers to complete recovery of the patient without the adverse outcome. The likelihood of a full and healthy recovery of the patient can be displayed on the user interface.

In some implementations, the system provides information indicative of a recommended treatment based on the likelihood of adverse outcome of the patient. For example, the system can classify patients into high-risk, medium-risk, and low-risk groups, e.g., based on the deviation 116a. For example, based on the position and the direction percentiles (computed in respect to a particular cohort, e.g., limited to a cardiac surgery cohort), each patient can be assigned a score or a rank. After assigning a score for each patent, the system can classify patients into different groups. For patients in the high-risk group who will likely develop adverse outcomes, the system can generate alert such that medical professionals can change a course of treatment, e.g., changing a drug to another drug, dosage of a drug, ventilation setting, and the like. In some implementations, the system computes a score for the patient based on the likelihood of adverse outcome of the patient. The score can be displayed on a user device such as the user device 150, e.g., a smartphone of a medical professional.

FIG. 2B is a flowchart of example process 250 for training a trajectory model. The process 200 will be described as being performed by a system of one or more computers programmed appropriately in accordance with this specification. For example, the system 100 of FIG. 1 can perform at least a portion of the example process. In some implementations, various steps of the process 200 can be run in parallel, in combination, in loops, or in any order.

The system obtains training patient data (252). The training patient data includes clinical laboratory results of a plurality of patients with one or more disease states. The clinical laboratory results include measurements of one or more of anion gap, blood-urea nitrogen, creatinine, hematocrit, glucose, platelet count, red cell distribution width, or white blood cell count.

The system processes the training patient data such that the clinical laboratory results of the plurality of patients are normalized by patient-specific baseline levels (254). The baseline levels of the clinical laboratory results represent the levels of a blood count or metabolic panel before the patient develops the disease state. The system can impute the patient-specific baseline levels based on data from multiple other patients. For example, a creatinine level of the patient can be imputed based on one or more other patients who have similar patient attributes, e.g., medical histories including historical clinical laboratory results and demographic information.

The system identifies one or more variables to be used in a phase plane (256). The system identifies high-dimensional clusters by applying unsupervised clustering (e.g., k-means clustering, hierarchical clustering, density-based clustering) to the plurality of training patient data. The clusters are associated with the one or more disease states. The system reduces dimensionality of the high-dimensional clusters and identifies the one or more variables that are significantly associated with the one or more disease states. To identify significantly associated variables, the system computes, for a different set of selected variables, a significance of a generalized linear model predicting the adverse outcome using selected variables in the plurality of training patient data and determines if the significance meets a threshold.

The system fits a trajectory model, using the one or more variables, to the plurality of training patient data (258). The trajectory model characterizes the one or more disease states by a recovery trajectory in the phase plane. In some implementations, the system fits the plurality of training data by an exponential decay of white blood cell count and a linear increase in platelet count.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a standalone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

### Examples

The following examples further describe examples of the trajectory model and uses of the trajectory model and do not limit the scope of the invention described in the claims.

### Methods

The following methods were used in the Examples below.

### Study Populations

Clinical laboratory data for the cardiac surgery cohort was retrieved from the Mass General Brigham Electronic Data Warehouse and Research Patient Data Registry for patients who underwent non-emergency^{1,2} cardiac surgery procedures at Massachusetts General Hospital (MGH) between 01-01-2016 and 31-21-2019. Additional adjudicated surgical and outcomes data was collected by the MGH Division of Cardiac Surgery for contribution to the national Society of Thoracic Surgeons (STS) database. Other cohorts were collated by querying the databases for patients admitted between 01-01-2016 and 31-21-2020 for one of the six surgical procedures: colectomy, Cesarean section, hip arthroplasty, hysterectomy, limb amputation, or Whipple surgery; or for patients who had one of five primary diagnoses during their stay: myocardial infarction (MI), stroke, Clostridium difficile colitis, COVID-19, and sepsis. Cohort summary statistics are given in FIG. 29.

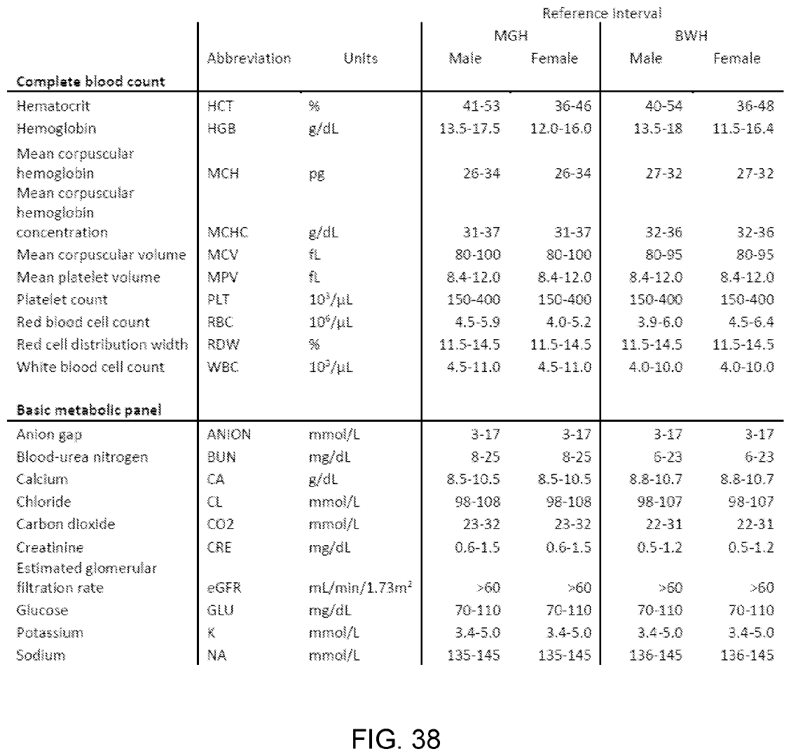

For all cohorts, demographics, outcomes, and all hematology (e.g., CBC) and clinical chemistry (e.g., BMP) test panels measured during the associated admission were retrieved. Analysis included all lab tests that were routinely collected at least once per day for hospitalized patients at the study hospitals (20 total measurements listed in FIG. 38). Adverse outcomes were defined as all-cause mortality within 30 days of discharge and long length of hospital stay (LOS) relative to typical cohort LOS: LOS>10 for surgeries with mean LOS<7 (Cesarean section, hip arthroplasty, hysterectomy) and LOS>14 otherwise.

### Cardiac Surgery Cohort

Data for the cardiac surgery cohort was collected from all adult Massachusetts General Hospital (MGH) patients who underwent cardiac surgery between 01-01-2016 and 31-12-2019 (N=6054). All blood count (CBC) and basic metabolic panel (BMP) measurements during the surgery-associated hospital stay were collected from the Partners Healthcare Electronic Data Warehouse (EDW) and Research Patient Data Registry (RPDR). Patient demographics, risk factors, surgical factors and post-operative outcomes were collected from a dataset adjudicated by the MGH Division of Cardiac Surgery for contribution to the national Society of Thoracic Surgeons (STS) database. Patients were excluded if their surgery was an emergency or salvage procedure, if they were discharged within 48 hours of surgery, or if they did not have at least 1 valid post-op CBC and BMP (post-exclusion N=4693). Analysis was initially limited to surgeries between 01-01-2016 and 30-09-2018 (N=3168, exploratory cohort). All key findings were then validated using surgeries from 01-10-2018 to 31-21-2019 (N=1525, validation cohort). An independent cohort was derived from cardiac surgeries at Brigham and Women's Hospital (BWH) between Jan. 1, 2016 and Sep. 30, 2019 (N=1988).

Reference intervals for blood count and metabolic panel biomarkers at both MGH and BWH are given in FIG. 38.

### Other Inflammatory Cohorts

Following analysis of the cardiac cohort, clinical data was collected from all adult MGH patients who underwent any of 6 major surgeries (colectomy, Cesarean section, hip arthroplasty, hysterectomy, limb amputation, Whipple procedure), or had any of 5 acute inflammatory diagnoses (myocardial infarction [MI], stroke, C. difficile colitis infection, or novel coronavirus infection [COVID-19], and sepsis), between 01-01-2016 and 31-12-2020. Patients discharged within 48 hours, without at least 2 CBCs, or those undergoing laparoscopic procedures were excluded, as were repeat visits from the same patient for the same event type. All stay associated CBCs and BMPs were collected, along with demographics, admission and discharge dates, and all-cause mortality, using RPDR and EDW.

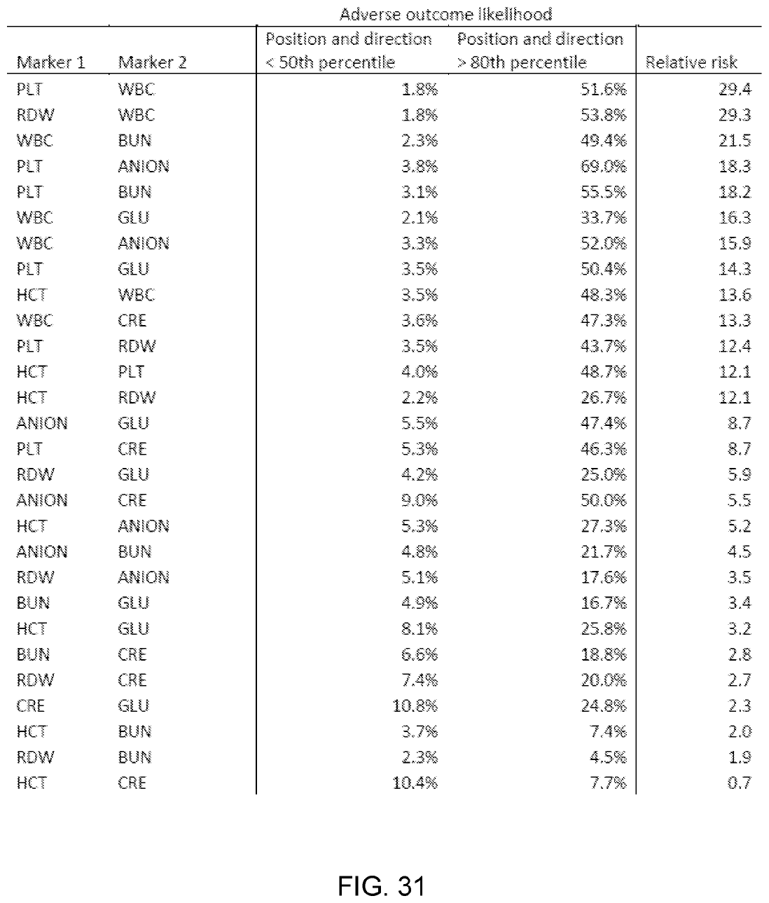

Following exclusions, each cohort was split evenly, with the earlier half (by diagnosis or surgery date) taken as the exploratory set, and the latter half taken as the validation set. Cohort sizes after each exclusion are show in FIG. 31.

Cohort exclusion criteria Patients were excluded if they were under 18 years old, had a less than 2 day associated hospital stay, or underwent a laparoscopic procedure (for surgical cohorts).

Some cohorts had additional inclusion/exclusion criteria specific to that setting:
Amputation: Surgeries were included only if they involved amputation of a leg (above or below knee), arm (above or below elbow), or of a whole foot or hand. Amputations of fingers or toes were not included.
Colectomy: Colectomy was defined as any major (invasive, non-laparoscopic) surgery of the small or large bowel and was predominantly small bowel resection or full or partial large bowel resection/colectomy.
Stroke: Stroke was defined as any diagnosis of a stroke or cerebrovascular accident.
C. difficile colitis: The colitis cohort was limited to patients with a diagnosis of C. difficile colitis or infectious colitis, or with a diagnosis of colitis, and a confirmed positive C. difficile toxin assay.
Sepsis: The sepsis cohort included any patients with diagnosis of sepsis regardless of whether the underlying infection/organism was specified and encompassed both mild and severe sepsis diagnoses.

The cardiac surgery cohort was derived from a manually curated dataset adjudicated by the Massachusetts General Hospital Division of Cardiac Surgery for contribution to the national Society of Thoracic Surgeons (STS) database. For all other cohorts, data was collected by filtering electronic health record databases for keywords associated with the surgery or diagnosis. For each cohort, terms were selected based on author clinical experience. For each cohort, a random sample of patient health records was manually checked to ensure that the database-listed diagnosis and procedures accurately reflected information in patient medical records. Due to the nature of the Partners Healthcare network databases, a small number of patients in each (non-cardiac surgery) cohort may not have received treatment exclusively at MGH, instead receiving part of their treatment at one of the other hospitals in the Partners Healthcare network.

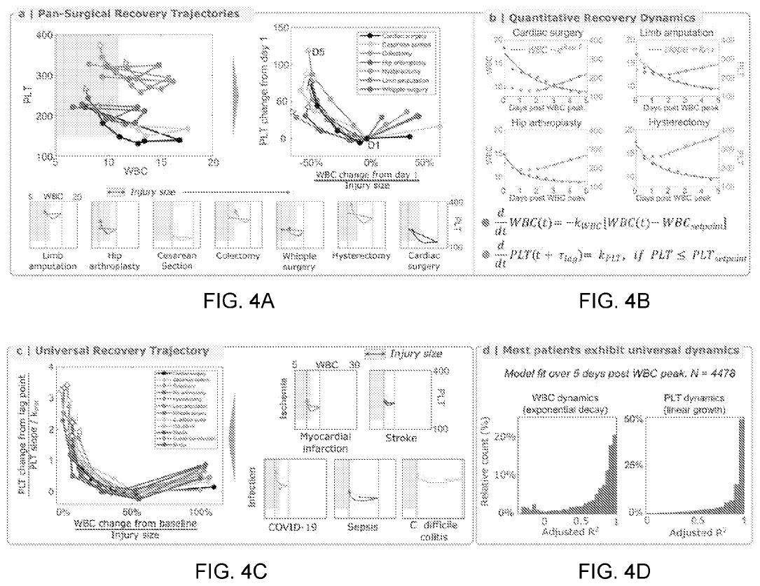

In addition to total cohort exclusions above, for accurate calculation of trajectories and model parameter estimation in FIG. 4, patients with 5 days of post-WBC peak data or more were included. To focus on situations where biological variation could be confidently distinguished from analytic variation, patients who did not have an inflammation-induced WBC increase of at least 2 units were excluded. The size of each of the cohorts after each exclusion are included in FIG. 31.

### Dimensionality Reduction and Trajectory Calculation

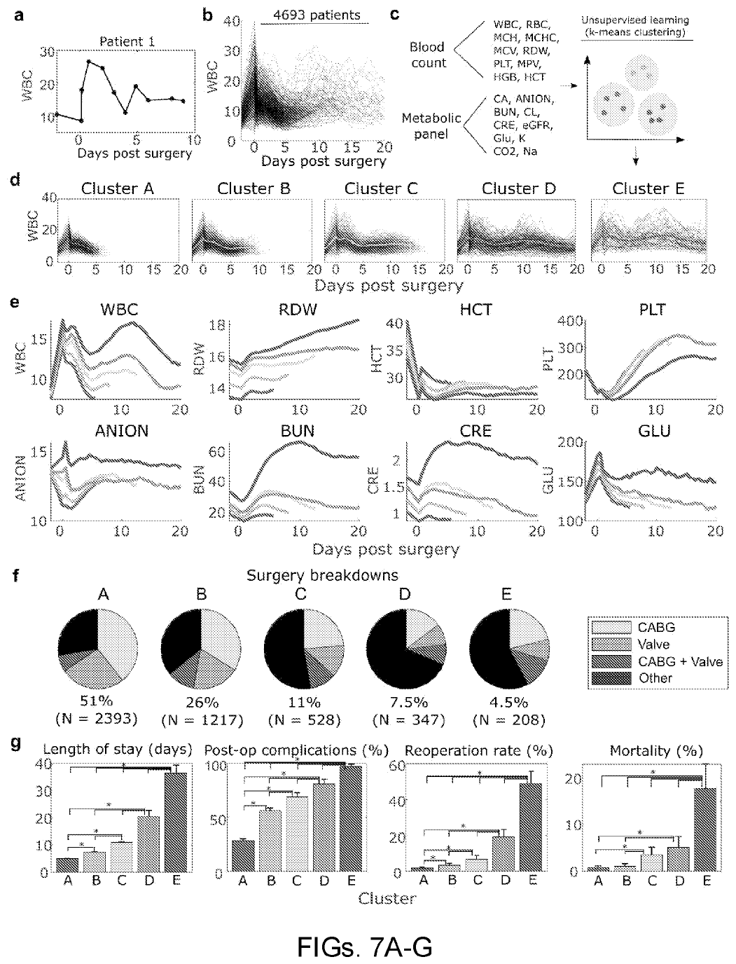

For preliminary analysis of inflammatory trajectories, clinical laboratory test results were interpolated and evenly sampled every 12 hours post-operation. Patient response clusters in the cardiac exploratory cohort (FIG. 7) were derived using k-means clustering³ applied to patient lab test measurements throughout the surgery-associated hospital stay. In FIG. 7, individual lab test results were interpolated (a), and these individual test trajectories (b) were considered for 20 standard complete blood count (CBC), basic metabolic panel (BMP), and other clinical laboratory tests (c). K-means clustering identified five distinct clusters (d, e), associated with significant differences (p<0.05) in surgery type (f) and post-operative outcomes (g). Day 0 corresponds to the first blood count measurement post-surgery, while day −2 corresponds to pre-operative measurements. Star (*) denotes values in (g) that were statistically significantly different (p>0.05) from each other.

Measurements were normalized (by pre-operative means), interpolated and sampled every 12 hours until discharge, with post-discharge values set to 0. The number of clusters (5) was the maximum number which resulted in all groups having more than 50 patients. Patients with fewer than 3 sets of measurements were not included when defining clusters but were assigned to their nearest group afterwards. Clinical tests which showed insignificant (<10%) variation across clusters were excluded, leaving 10 measurements. Given the high correlation (Pearson coefficient>0.9) between HCT, HGB and RBC, only HCT was included in the final clustering, leaving 8 measurements: anion gap (ANION), blood-urea nitrogen (BUN), creatinine (CRE), hematocrit (HCT), glucose (GLU), platelet count (PLT), red cell distribution width (RDW), and WBC. Statistical significance of trajectory clusters was tested through comparison to synthetic data, using a method outlined by Liu et al³.

Dimensionality was further reduced by identifying the optimal pair of results (from the 28 possible combinations) for risk stratification. High-performance pairs were identified by considering the significance (p-value, χ² test) of a generalized linear model predicting mortality using the interpolated post-op day 1 measurements from each biomarker pair. Clinical and physiologic interpretability were also subjectively considered, validating the choice of WBC-PLT for 2D distillation of the high-dimensional dataset.

### WBC-PLT Phase Plane Analysis

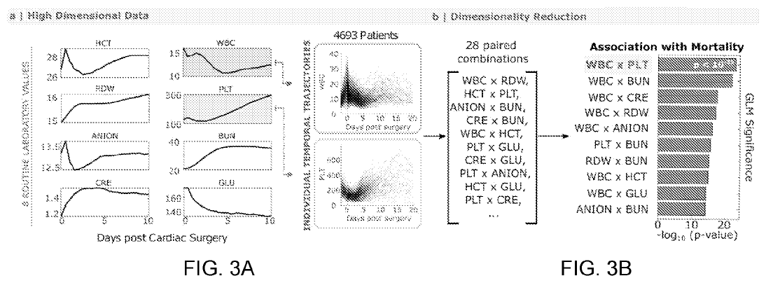

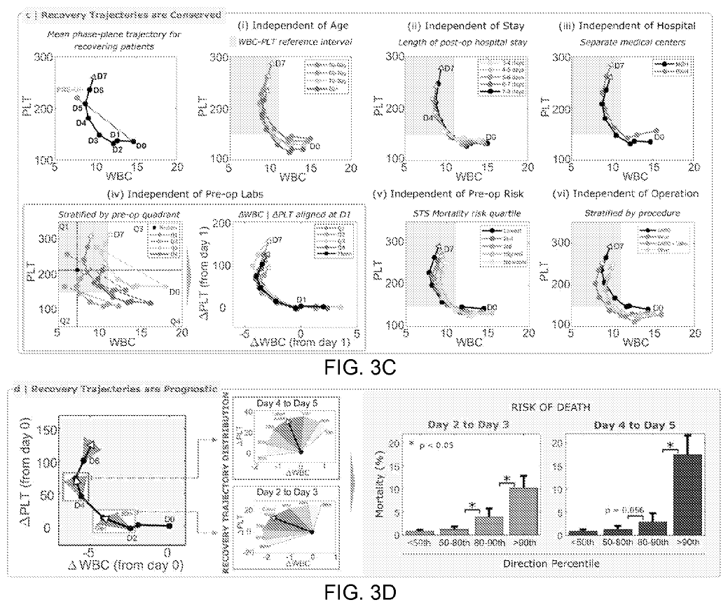

In the exploratory cardiac surgery cohort, dynamics in the WBC-PLT phase plane were analyzed by calculating the mean WBC-PLT response for patients who survived with hospital stays of less than 14 days. Mean responses were then calculated for subsets of this cohort stratified by age, length of hospital stay, medical center, pre-op lab values, pre-op risk evaluations, and surgery type (FIG. 3C). Preliminary risk evaluations (FIG. 3D) were calculated by comparing the angle of deviation of validation cohort patients against the reference trajectory to the associated deviation distribution in the exploratory cohort. Angles against the reference trajectory were calculated using the WBC and PLT change over the past 24 hours, after normalization by their pre-operative means.

### Mathematical Modeling of a Recovery Trajectory

Models of exponential decay and linear growth were fit to WBC and PLT counts. To account for differences in precise timing of the initiation of the acute inflammatory response, patients were aligned according to the time of their peak WBC count: in the 3 days after surgery (surgery cohorts), in the 3 days after admission (ischemia), or over their entire stay (infection). An exponential decay model (y=a+be^{-ct}; parameters: a, b, c) and a linear model (y=d+f*t; parameters: d, f) were fit to patient WBC and PLT counts over the first 5 days after peak WBC. The PLT model was fit after a lag τ, which allows for potential delay between peak WBC and the start of PLT growth. The favorable response for each cohort (FIG. 4) was calculated as the mean WBC-PLT trajectory after peak WBC for all patients without adverse outcomes who had at least 5 days of WBC and PLT measurements after peak WBC and whose peak WBC was at least

$$2 \cdot \frac{1 {0}^{3} cells}{μ l}$$

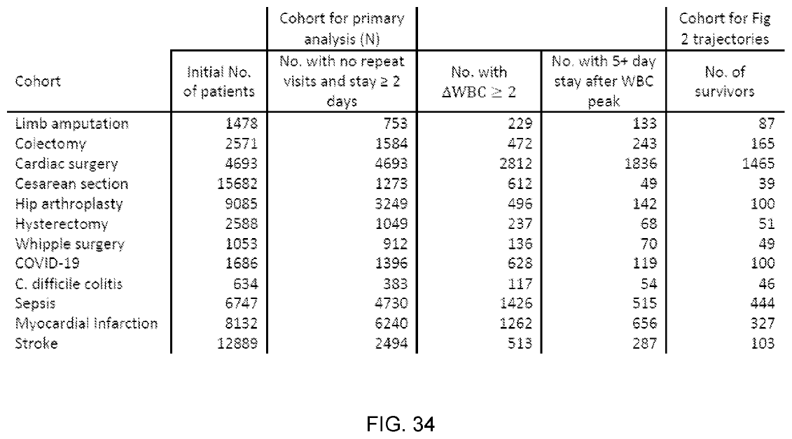

above their initial WBC (see FIG. 34 for cohort sizes).

### WBC-PLT Trajectory-Based Risk Analysis

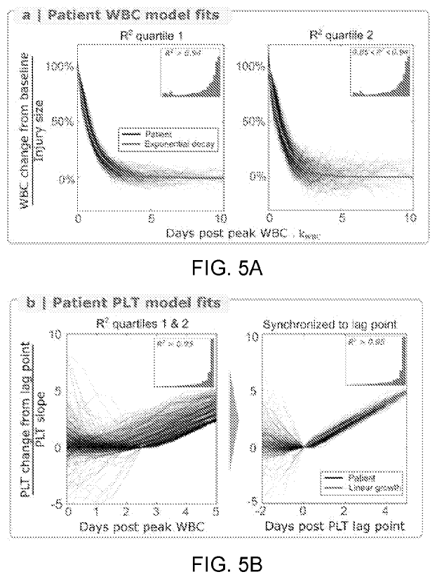

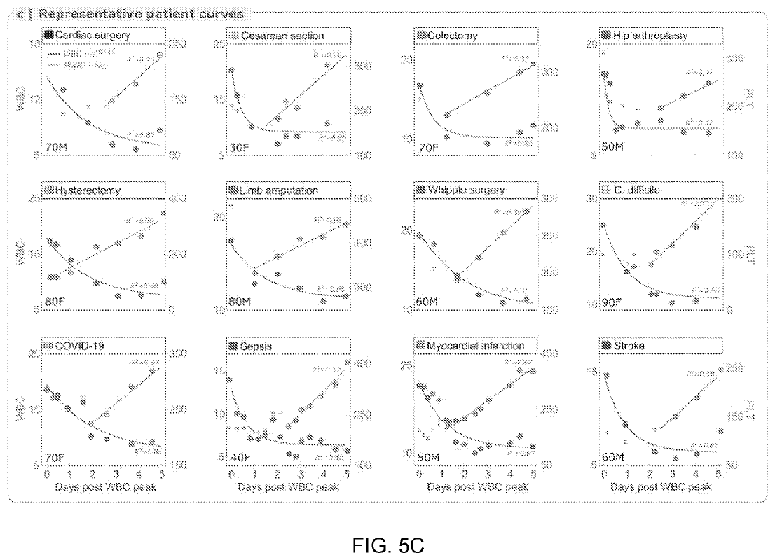

Patient risk of adverse outcomes was calculated for an independent validation cohort of patients by comparing a patient's position and movement direction in the WBC-PLT phase-plane to the mean WBC-PLT trajectory calculated for patients in an independent exploratory cohort without adverse outcomes. Positional risk was calculated as the distance from the mean trajectory, after normalizing WBC and PLT by baseline means and treating WBC below and PLT above the mean as contributing zero distance. Directional risk was calculated as the angle between the patient's normalized daily WBC and PLT change vector and the normalized mean WBC-PLT trajectory change vector. Each patient's relative position and direction were converted to percentiles relative to distributions in exploratory cohorts. All thresholds were determined using the exploratory cohorts, and all risk stratifications based on these thresholds were calculated for the validation cohorts. Choices of percentile thresholds in FIG. 5 were made from analysis of only the cardiac surgery exploratory cohort. Because of uncertain inflammatory event timing in the infection cohorts, risk calculations were performed after patient alignment using the timing of peak WBC count within the first 72 hours after admission.

For completeness, risk stratification of the COVID-19 cohort was calculated without aligning the cohort based on their peak WBC values (within 72 hours of admission), as was done in FIG. 5. While the exact prevalence rates differed from the aligned results in FIG. 3, risk stratification remained significant regardless of this alignment as shown in FIG. 18.

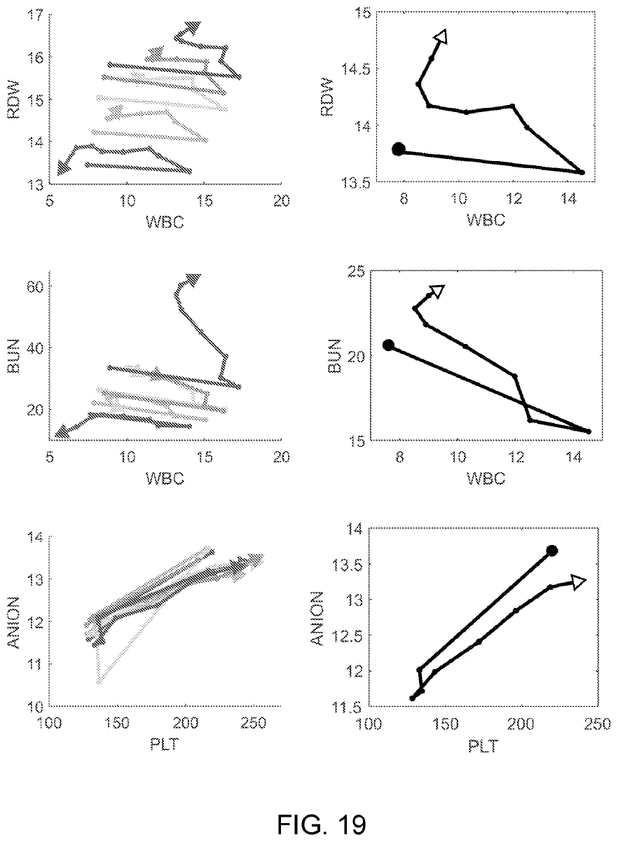

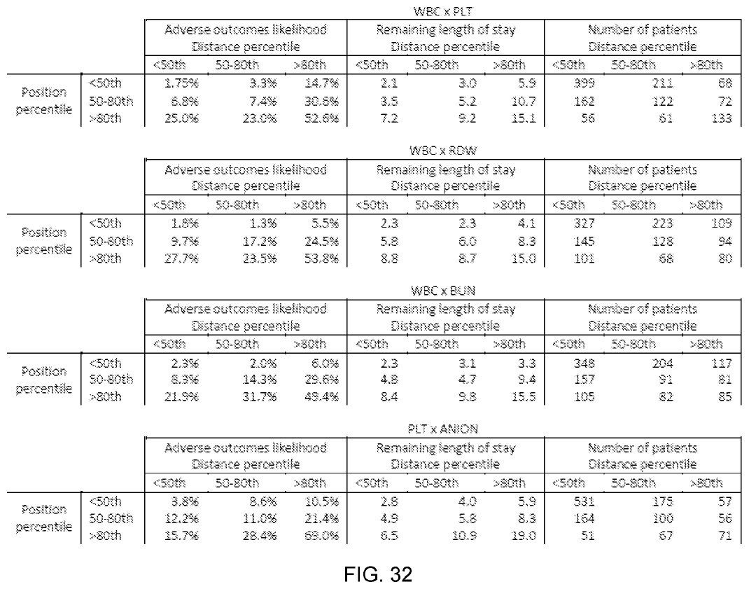

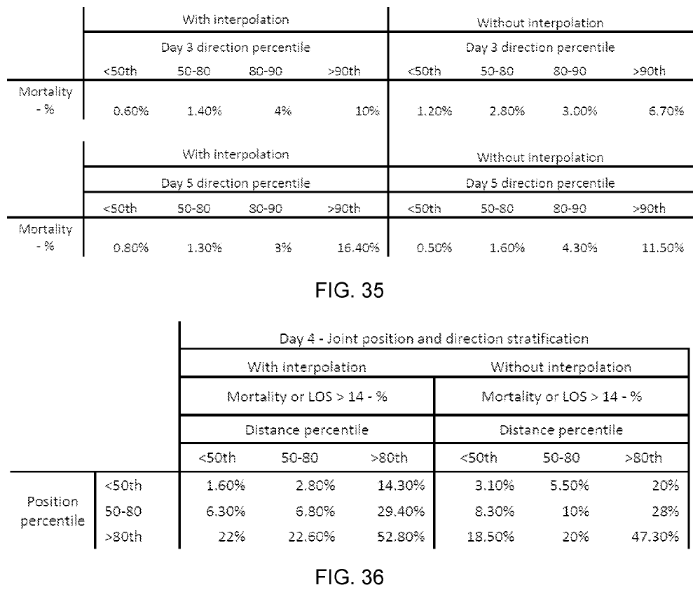

Risk analysis in the cardiac surgery cohort was also performed using other high-significance test result pairs (WBC-RDW, WBC-BUN, PLT-ANION), with WBC-PLT maintaining the strongest risk stratification (FIGS. 31-32) and illustrating a more robust recovery trajectory (FIG. 19). FIG. 19 shows mean trajectories for each cluster (left) and for patients with good outcomes (right) are given for three alternate combinations of test results: WBC×RDW, WBC×BUN, and PLT×ANION. Each trajectory is from pre-op to day 7, with spacing between dots equal to 1 day. Risk stratification in the cardiac surgery cohort was also preserved when performed using non-interpolated laboratory values (FIGS. 35-36), with patient position and direction at a given time calculated using the last two available blood counts (excluding repeats). FIG. 35 shows comparison of mortality risk for cardiac surgery cohort stratified by direction percentiles, using interpolated and non-interpolated laboratory value trajectories. FIG. 36 shows mortality or long stay risk for cardiac surgery cohort stratified by joint position and direction percentiles, using non-interpolated laboratory values.

### Statistical Analysis

Statistical analysis was performed in MATLAB. For all continuous variables, unless otherwise noted, we report means and use analysis of variance (ANOVA) for population comparisons. For categorical variables we report percentages and use a chi-square test for population comparisons. Thresholds for statistical significance were set at p=0.05. For event rates, confidence intervals were calculated assuming binomial distributions. WBC and PLT trajectories were interpolated and evenly sampled every 12 hours during patient admission.

### Example 1. Multivariate Test Result Trajectories During Inflammatory Recovery

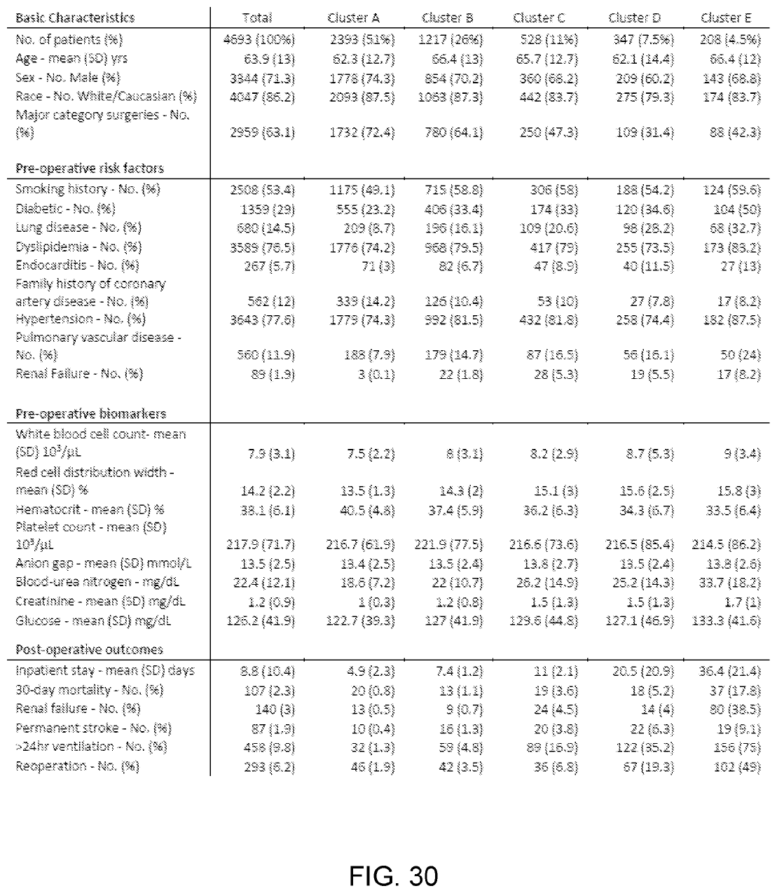

We considered the inflammatory response to non-emergency cardiac surgery because it involves (1) a significant inflammatory challenge (sternotomy with cardiopulmonary bypass) with well-defined timing and consistent magnitude and (2) relatively stable baseline patient conditions across the cohort. We studied 4693 patients at Massachusetts General Hospital (MGH): an exploratory cohort with surgery between 01-01-2016 and 30-09-2018, N=3168, and a validation cohort with surgery between 01-10-2018 and 31-12-2019, N=1525 (FIG. 29). In this example, adverse outcomes were defined as death or a post-operative hospital length of stay (LOS) greater than 14 days. As shown in FIG. 3A, reduced-dimension analysis of trajectories of complete blood count (CBC) and basic metabolic panel (BMP) measurements, using unsupervised clustering, retained significant association with patient outcome and identified high-risk and low-risk subsets with 22-fold mortality risk differences (0.8% to 17.8%) (FIG. 7, FIG. 30). Multiple individual markers, including WBC and PLT, were significantly auto-correlated and cross-correlated (FIGS. 31-32), suggesting robust co-regulation of blood cell populations and serum markers, and implying that reduced-dimension phenotypes would retain sufficient information to identify common features of favorable responses.

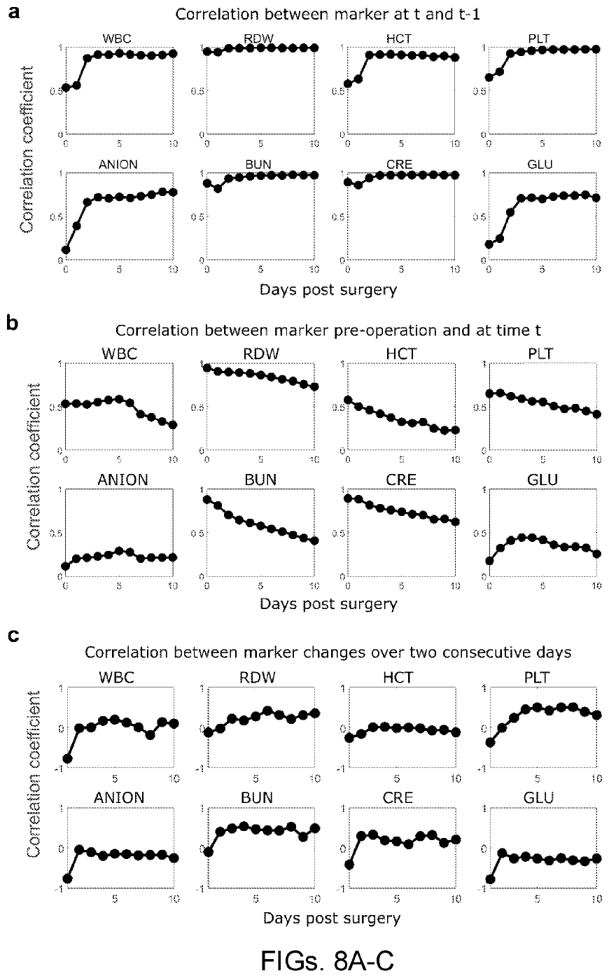

In FIG. 8, correlation coefficients were given for 8 tests (WBC, RDW, HCT, PLT, ANION, BUN, CRE, GLU) throughout recovery from cardiac surgery. Correlations were provided between values over consecutive days (a), between current and baseline values (b) and between marker changes over consecutive days (c). All markers exhibited high autocorrelation over consecutive days, with four markers (RDW, PLT, BUN, CRE) having correlations continually above 0.9, reflecting slower dynamics than the other four markers. Three of the markers (WBC, ANION, GLU) also exhibited a type of ‘memory’, where correlation of day 5 values with pre-operative values was higher than correlations in the preceding days. This pattern might reflect a homeostatic memory, whereby patients return to their baseline. Four markers (RDW, PLT, BUN CRE) showed high correlations between changes over consecutive days, reflecting a high momentum for these markers.

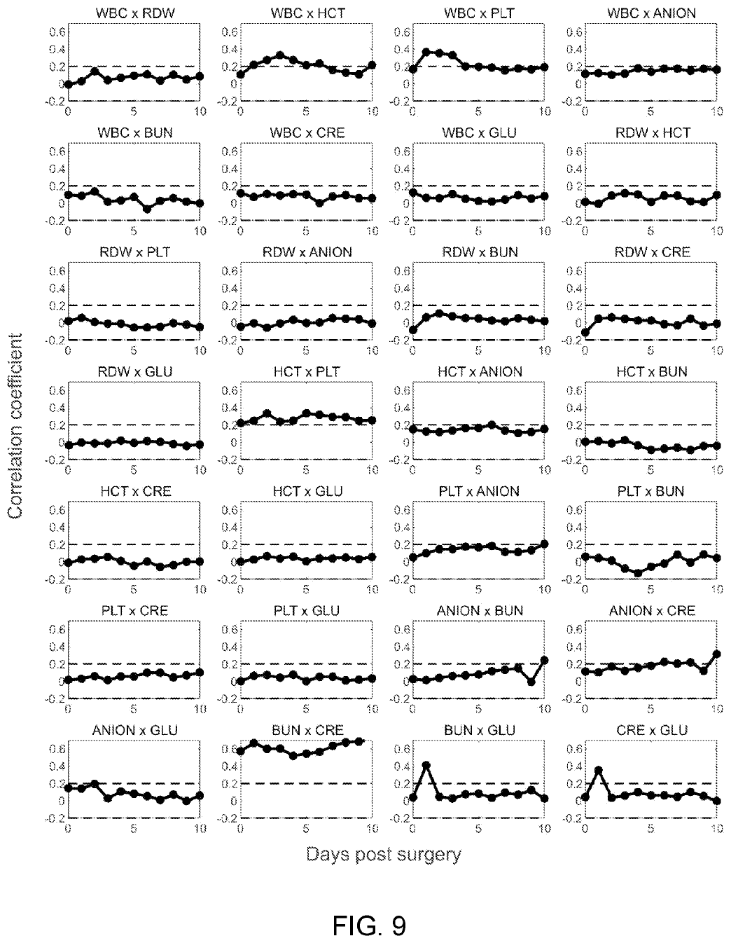

FIG. 9 shows cross-correlation coefficients for daily changes in 8 biomarkers (WBC, RDW, HCT, PLT, ANION, BUN, CRE, GLU) throughout recovery from cardiac surgery. Coefficients were the correlations between the change in each pair of results over the preceding 24 hr period. Most result pairs showed low cross-correlation, except for blood cell populations (WBC×HCT, WBC×PLT, HCT×PLT), suggesting strong coregulation of blood cell populations, and renal function tests (BUN×CRE).

### Example 2. WBC-PLT Trajectory During Inflammatory Recovery from cardiac surgery

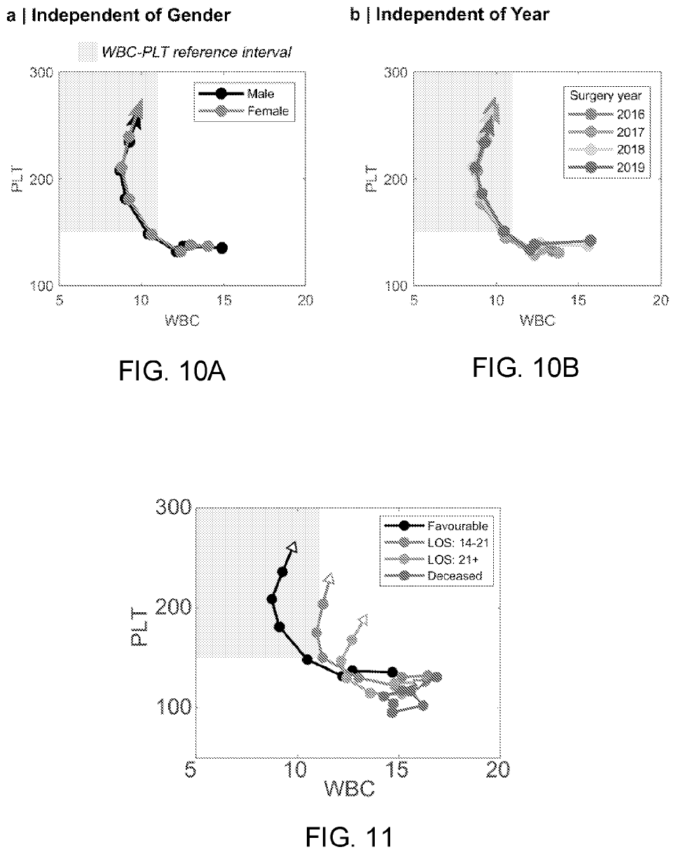

To reduce dimensionality and retain interpretability⁴⁻⁸, we performed phase plane analysis, a powerful approach commonly used in engineering and applied mathematics to understand the quantitative behavior of dynamical systems by analyzing subsets of system variables as a function of time⁹. We focused on the WBC-PLT plane because WBCs and PLTs play primary roles in inflammation^{10, 11, 2}, and the WBC-PLT combination provided at least as strong statistical association with outcomes as other pairwise combinations (FIG. 3B, FIGS. 31-32). Patient WBC-PLT trajectories showed that cardiac surgery is a dramatic inflammatory perturbation, moving more than 95% of patients outside the reference intervals for WBC or PLT (FIG. 3C). To investigate whether cardiac surgery patients who recover well follow homogeneous or heterogeneous paths, we analyzed WBC-PLT trajectories for patients who survived and had LOS<14 days^{1,2}. Individual patient trajectories reflected divergent baseline characteristics and significant variation in clinical events, especially over short periods of time (FIG. 3A), but the shape of the average WBC-PLT recovery trajectory was nevertheless conserved and robust to variation in patient age, LOS, hospital of service, baseline WBC and PLT, standardized pre-operative risk assessment^{1,2}, operation type, patient gender, year of surgery, and more (FIG. 3C, FIG. 9). FIG. 10A shows that the WBC-PLT recovery trajectory is robust, independent of gender. FIG. 10B shows that the WBC-PLT recovery trajectory is robust, independent of year.

Patients with poor outcomes did not demonstrate this WBC-PLT trajectory shape (FIG. 11). FIG. 11 shows mean trajectories for patients who survived with post-op hospital stays of 2-3 weeks, greater than 3 weeks, and for patients who did not survive. For comparison, the reference trajectory from FIG. 3C was also included as a favorable trajectory. Unlike the results in FIG. 1c, there is much lower coherence between the mean trajectories of patients with different unfavorable outcomes, with each set of unfavourable outcomes being distinct from the favourable outcome trajectory. Thus, WBC and PLT populations were, on average, co-regulated in a consistent way during the resolution phase of an effective inflammatory response to cardiac surgery.

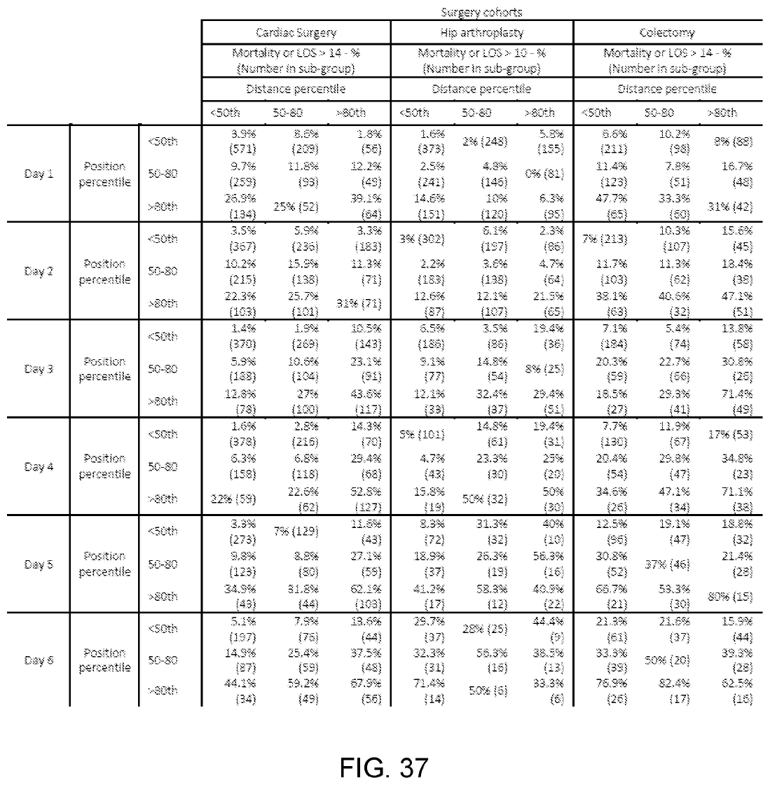

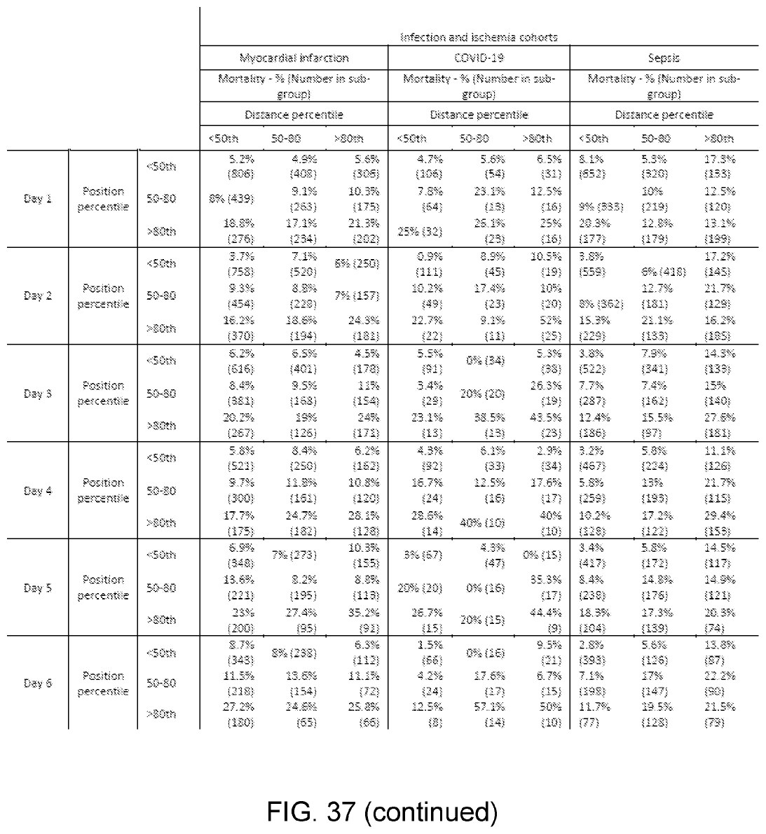

Deviation from the mean recovery trajectory is associated with adverse outcomes in a separate validation cohort. In FIG. 3D, the mean trajectory along with the 50^{th}, 80^{th} and 90^{th} percentiles for daily directional changes are shown. Deviation from the direction of the mean trajectory is associated with significant (star: *, p<0.05) increased risk of death for patients above the 90^{th} percentile compared to those below the 50th: 14× (p<0.001; confidence interval (CI): 8.0-24.1, 0.7% to 10.2%) on day 3 after surgery and 22× (p<0.001; CI: 11.7-39.8, 0.8% to 17.4%) on day 5 (error bars denote 95% CI). Percentile thresholds were calculated in an exploratory cohort and outcome rates in an independent validation cohort. FIG. 19 shows trajectories for alternate test result pairs. Trajectories for WBC lineage (neutrophils, lymphocytes, etc.) are shown in FIG. 37.

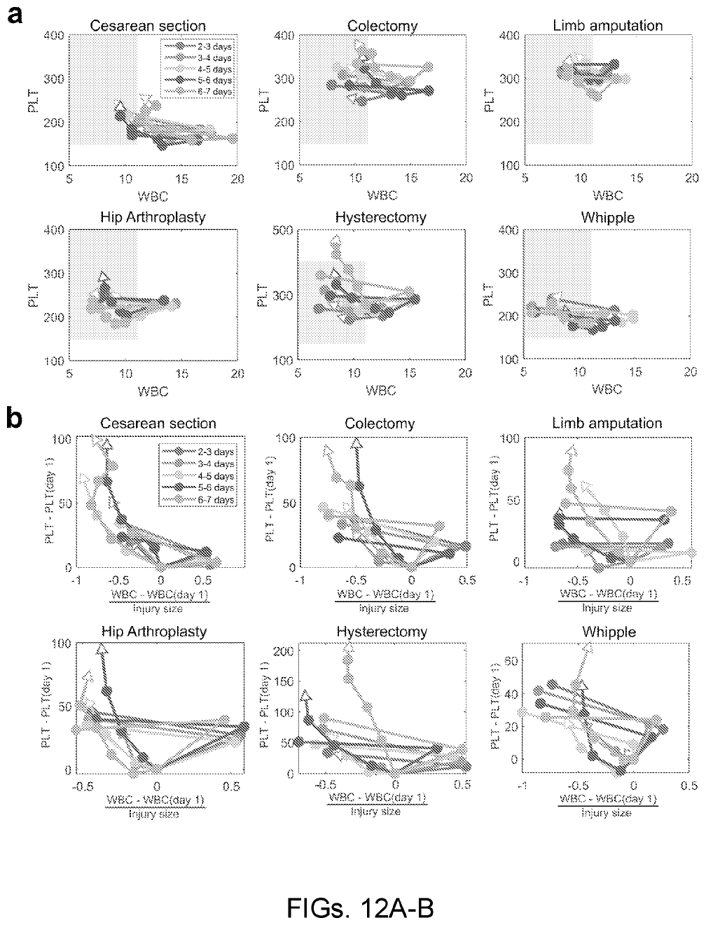

The persistence of this WBC-PLT trajectory shape motivated investigation of cohorts undergoing six other major surgeries: Cesarean section, colectomy, hip arthroplasty, hysterectomy, Whipple procedure, and limb amputation (FIG. 29). As shown in FIG. 4A, the average favorable WBC-PLT recovery trajectories of all 7 cohorts were similar after adjusting for baseline WBC and PLT and the magnitude of initial WBC response. This average WBC-PLT recovery shape was robust to the large differences in demographics, baseline health, and surgical indication across these cohorts (FIG. 12). FIG. 12 shows results for patients with LOS of 2-3, 3-4, 4-5, 5-6, and 6-7 days, without normalization (a), and with normalization (b) based on post-op day 1 WBC and PLT, and injury size (post-op WBC−pre-op WBC). While patients with different LOS had different baseline WBC-PLT and injury size, the shape of the mean normalized trajectories were consistent for each of the 6 surgery cohorts.

### Example 3. WBC-PLT Recovery Shows Exponential WBC Decay and Linear PLT Growth

Across the 7 surgical cohorts, WBC recovery dynamics could be approximated as an exponential decay from the maximum post-op WBC (WBCₘₐₓ) toward the patient's pre-inflammation baseline or homeostatic setpoint (WBCₛₑₜₚₒᵢₙₜ) with a decay constant of k_{WBC}_{_{decay}} days⁻¹ (FIG. 4B):

$$WBC ( t ) = ( {WBC}_{max} - {WBC}_{setpoint} ) {e}^{- {k}_{{WBC}_{decay}} \cdot ( t - {t}_{{WBC}_{max}} )} , for t \geq {t}_{{WBC}_{max}} , where {WBC}_{max} = WBC ( {t}_{{WBC}_{max}} )$$

Starting τ_{PLT delay} days after t_{WBC}_{ₘₐₓ}, PLT counts grew approximately linearly with a slope

$${k}_{{PLT}_{g r o w t h}} \frac{1 {0}^{3}}{μ l \cdot day}$$

### (FIG. 4B):

$$PLT ( t ) = PLT ( {t}_{{WBC}_{max}} + {τ}_{PLT\_delay} ) + {k}_{{PLT}_{growth}} \cdot ( t - {t}_{{WBC}_{max}} - {τ}_{{PLT}_{delay}} ) , for t \geq ( {t}_{{WBC}_{max}} + {τ}_{{PLT}_{delay}} ) .$$

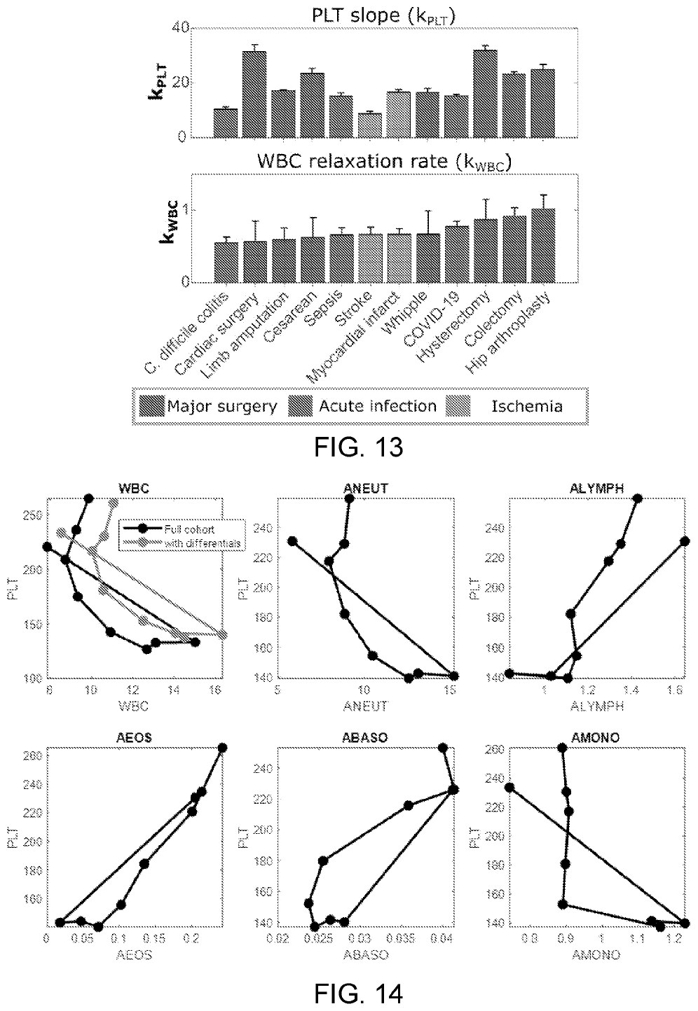

A simple mechanistic model consistent with these observed recovery dynamics is one of a homeostatic feedback control system^{12,13} where (i) excess WBCs are removed at a constant rate per WBC, with negligible additional WBCs introduced into the circulation, (ii) PLTs are produced at a constant rate with negligible consumption starting approximately 2 days later, and (iii) both processes continue at least until the patient's baseline setpoints are reached. Model fits of k_{WBC_decay} across the cohorts (FIG. 13) suggested that WBCs were removed at random after circulating for an average duration of ˜1-2 days after t_{WBC}_{ₘₐₓ}, corresponding to a half-life of 0.7-1.4 days. PLT growth started about two days after the peak WBC, continuing until reaching or exceeding the setpoint. In the cardiac surgery cohort where more data were available, most recovering patients appeared to overshoot their baseline PLTₛₑₜₚₒᵢₙₜ (FIG. 3C). WBC population dynamics were driven primarily by neutrophils (FIG. 14).

### Example 4. WBC-PLT Recovery Trajectory in Infection and Ischemia

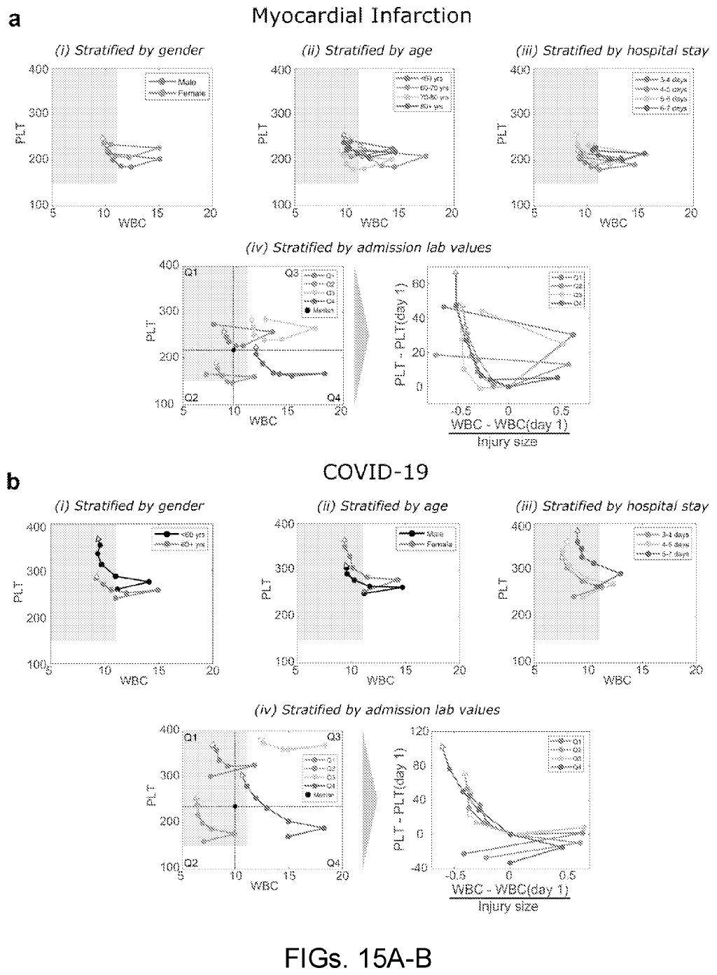

Given the resilience of the conserved recovery trajectory shape across a diverse range of surgical trauma, we investigated whether the pattern persisted in non-traumatic inflammatory conditions: three types of infection (COVID-19, Clostridium difficile colitis, and sepsis) and two types of ischemia (myocardial infarction and stroke). As shown in FIG. 4C, the same basic response shape was identified. WBC and PLT kinetics for the average recovering patient in each cohort were well-explained by exponential WBC decay and delayed linear PLT growth. As in the surgical cohorts, this characteristic response was conserved when the cohorts were stratified by gender, age, LOS, and baseline WBC-PLT (FIG. 15A for myocardial infarction cohort and FIG. 15B for COVID-19 cohort). To reveal characteristic features of WBC and PLT dynamics intrinsic to all inflammatory recoveries, we adjusted for the differences in baseline and rate parameters by non-dimensionalizing¹⁴ the model using the following dimensionless variables (w, p, t*):

$$\begin{matrix}w = \frac{WBC - {WBC}_{setpoint}}{{WBC}_{max} - {WBC}_{setpoint}} \\ p = \frac{PLT - PLT ( {t}_{{WBC}_{max}} + {τ}_{{PLT}_{delay}} )}{( \frac{{k}_{{PLT}_{production}}}{{k}_{{WBC}_{decay}}} )} \\ {t}^{*} = ( t - {t}_{{WBC}_{max}} ) \cdot {k}_{{WBC}_{decay}} , for t \geq {t}_{{WBC}_{max}}\end{matrix}$$

This variable transformation (FIG. 4C) made clear that WBC-PLT dynamics during recovery from all of these inflammatory states were well-characterized as exponential WBC decay and delayed linear PLT growth:

$$\begin{matrix}w ( {t}^{*} ) = {e}^{- {t}^{*}} , for {t}^{*} \geq 0 \\ p ( {t}^{*} ) = {t}^{*} - {τ}_{{PLT}_{delay}} \cdot {k}_{{WBC}_{decay}} , for {t}^{*} \geq {τ}_{{PLT}_{delay}} \cdot {k}_{{WBC}_{decay}}\end{matrix}$$

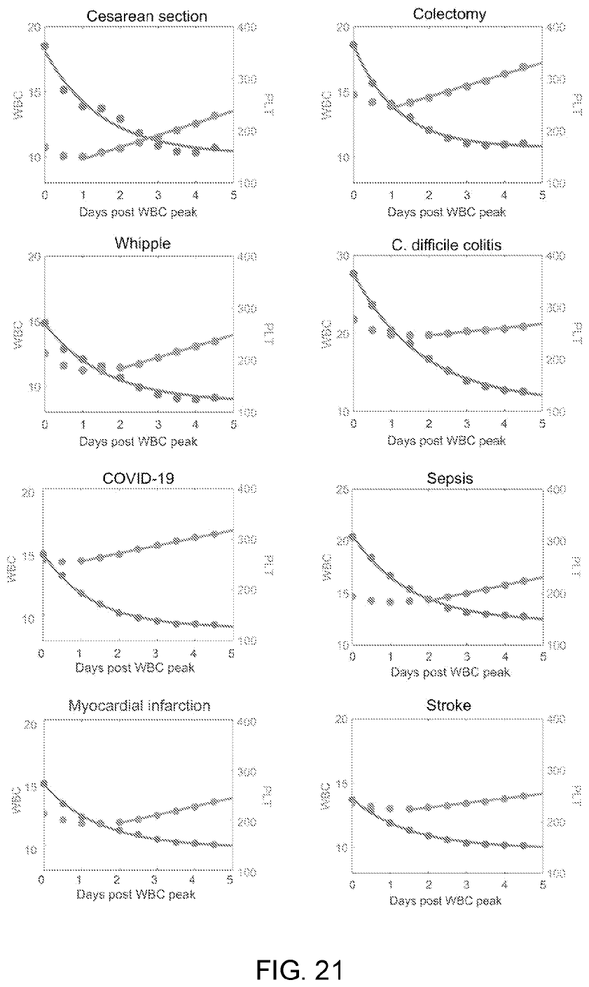

For all patients with sufficient data across all inflammatory cohorts, this trajectory model fitted individual patient WBC-PLT trajectories with a median adjusted R² of 0.84 for WBC and 0.92 for PLT (FIG. 4D). FIG. 21 shows model fits for the other 8 cohorts. FIG. 13 shows fitted model parameters (k_{WBC}, k_{PLT}) for all cohorts.

As shown in FIG. 5, typical recovering patients followed this recovery shape, regardless of age or condition. As shown in FIG. 5A, across all 12 cohorts, individual patient WBC recovery dynamics were well-approximated by the model with median adjusted R²=0.85, as are individual PLT recovery dynamics (see FIG. 5B) with a median adjusted R² of 0.95 following a variable delay. As shown in FIG. 5C, WBC-PLT dynamics for representative patients followed the model. Representative patients were those with each inflammatory condition whose WBC-PLT data had model fits closest to the cohort median adjusted R². These patients exhibited exponential WBC decay and delayed linear PLT increase during recovery despite significant differences in gender and age (bottom left of each panel), inflammatory stimulus, CBC frequency, and magnitude of WBC and PLT count changes.

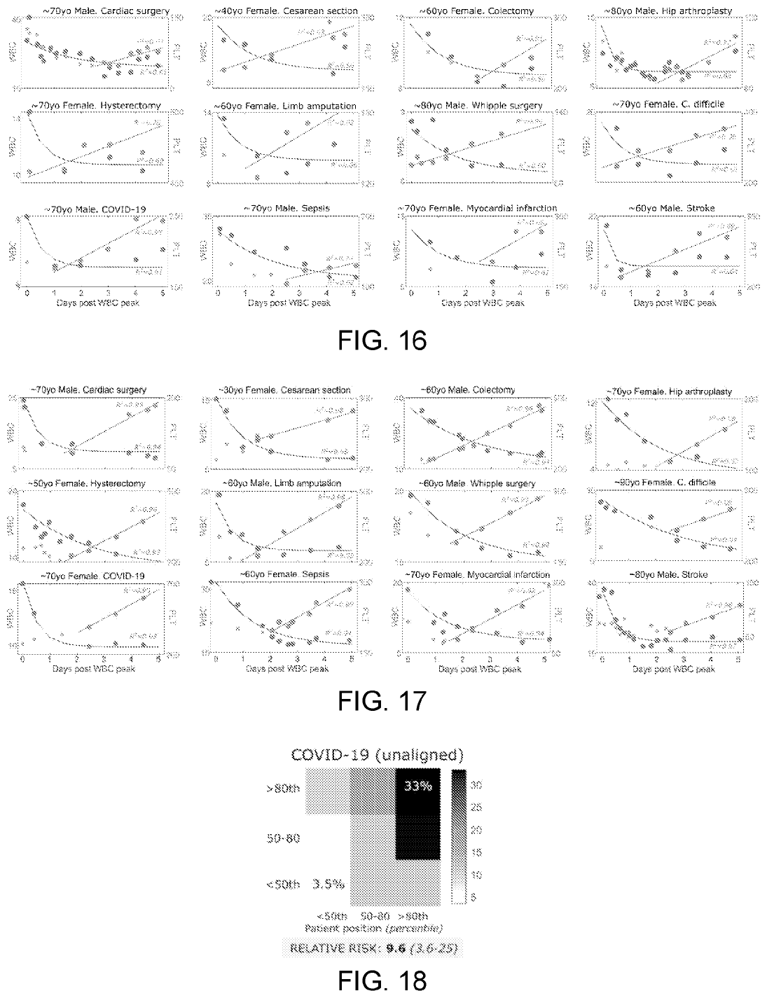

Additional individual patient trajectories for patients whose raw WBC-PLT data had model fits near the 25^{th} and 75^{th} percentiles of the cohort adjusted R² distributions are shown in FIG. 16 and FIG. 17.

FIG. 16 shows example patient trajectories and model fits for patients whose raw data had goodness of fit with adjusted R² near the 25^{th} percentile (poorest fitting quartile). Raw patient data and corresponding WBC and PLT model fits were given for a patient in each cohort whose model fit had an adjusted R² closest to the 25^{th} percentile for PLT R² (0.6) and WBC R² (0.82). Despite having poorer fits than ˜75% of the cohort, the pattern of WBC exponential decay and delayed linear PLT increase was apparent.

FIG. 17 shows example patient trajectories and model fits for patients whose raw data had goodness of fit with adjusted R² near the 75^{th} percentile (best fitting quartile). Raw patient data and corresponding WBC and PLT model fits were given for a patient in each cohort whose model fit had an adjusted R² closest to the 75^{th} percentile for PLT R² (0.98) and WBC R² (0.94).

### Example 5. WBC-PLT Trajectory-Based Patient Risk Assessment

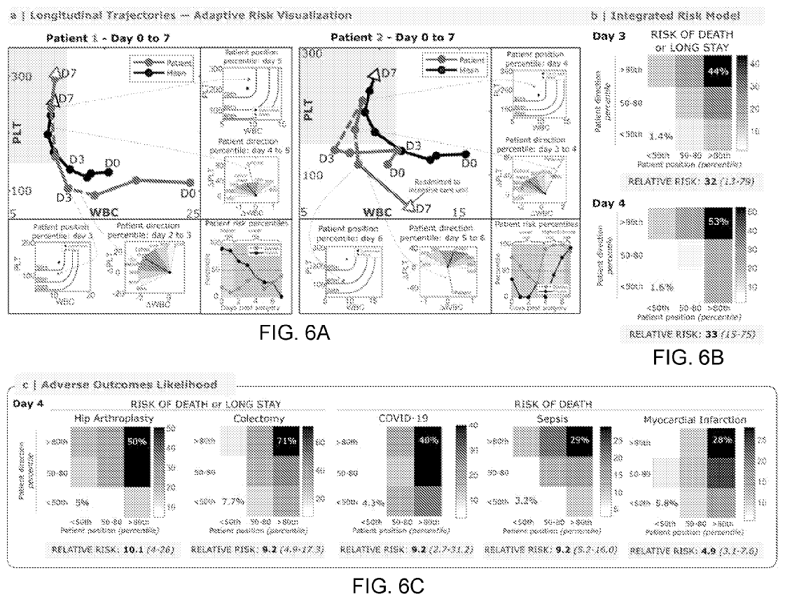

Deviation from the direction of the favorable recovery trajectory shape was associated with elevated risk of adverse outcomes following cardiac surgery (FIG. 3D). We further considered that WBC-PLT position provided an integrated trajectory-based risk assessment. As shown in the left panel of FIG. 6A, we assessed the individual patient risk on the hospital course of a 55-year-old female with a prior mechanical aortic valve replacement and high STS pre-operative risk of mortality (PROM)^{1,2}(10%, 95^{th} percentile). Repeat sternotomy for aortic valve replacement was complicated by intra-operative bleeding and hypotension requiring rapid transfusion and resuscitation, but the post-op recovery was otherwise smooth. Daily comparison of the patient's WBC-PLT position and direction yielded a high initial risk. This high risk steadily declined as the patient's WBC normalizes, and the trajectory of the patient merged quickly with the reference shape of (favorable) recovery.

In contrast, the right panel of FIG. 6A shows the clinical course for an 84-year-old female with a history of angina and moderate PROM (2.6%, 75^{th} percentile) who initially recovered well after successful three-vessel coronary artery bypass grafting. Counts normalized by post-op day 4, but the rising WBC and subtly declining PLT thereafter nevertheless correspond to a marked deviation from the favorable trajectory and a sharp rise in directional risk. On post-op day 6, she developed fever and shortness of breath leading to a diagnosis of pulmonary embolism and initiation of heparin. A precipitous interval decline in PLT on day 7 led to diagnosis of heparin-induced thrombocytopenia, intensive care unit readmission, and a subsequent prolonged hospital stay of one month.

These two cases shown in FIG. 6A demonstrate how the WBC-PLT recovery shape can be used to identify high-risk patients who recover smoothly and to detect smoldering issues, which precede adverse events.

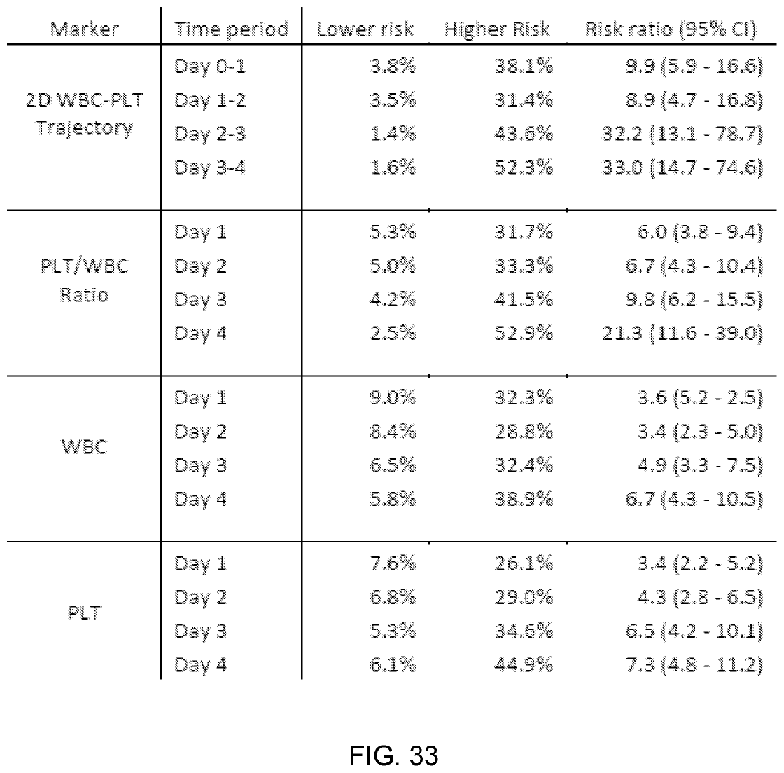

As shown in FIG. 6B, for the cardiac surgery cohort, patients outside the 80^{th} percentiles of position and direction had a relative risk of adverse outcomes of 32 on day 3 (CI: 13-79, 1.4% to 44%) and 33 on day 4 (CI: 15-75, 1.6% to 53%). Extending this analysis to the other inflammatory scenarios studied revealed significant relative risks: 9.2 (2.7-31.2, 4.3% to 40%) for death from COVID-19, 4.9 (3.1-7.6, 5.8% to 28%) for death following myocardial infarction, and 10.1 (CI: 4-26, 5% to 50%) for hip replacement, with all results from out-of-sample testing (FIG. 6C). In the validation cardiac surgery cohort, 97% (29/30) of deaths occurred in patients whose day 4 position or direction was above the 80^{th} percentile, with 83% (25/30) occurring in patients with both position and direction above the 80^{th} percentile. Corresponding specificities were 71% (865/1226) and 92% (1125/1226). Further risk analysis in the cardiac surgery cohort illustrated that consideration of the one-dimensional PLT/WBC ratio, or of PLT and WBC in isolation produced a significantly weaker risk stratification than the dynamic two-dimensional approach (FIG. 33)

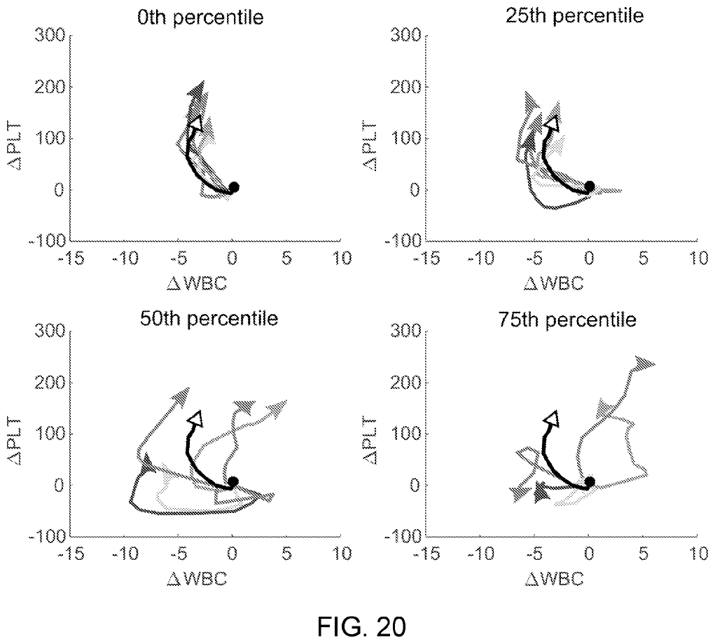

FIG. 20 shows example cardiac surgery WBC-PLT trajectories stratified by average degree of deviation for the full cohort. The 5 patient trajectories closest to the 0^{th}, 25^{th}, 50^{th} and 75^{th} percentile of average deviation (from day 1 to day 7) from the mean WBC-PLT trajectory were given, from post op day 1 to day 7. The 0^{th} and 25^{th} percentile trajectories adhered closely to the mean trajectory. The 50^{th} percentile exhibited high variance early on, but eventually adhered to the shape of the mean trajectory. No consistent patterns were seen in the 75^{th} percentile trajectory.

FIG. 32 shows patient outcomes stratified by day 4 position and direction percentiles for alternate 2D test result combinations, including WBC×RDW, WBC×BUN, and PLT×ANION, in the cardiac surgery cohort.

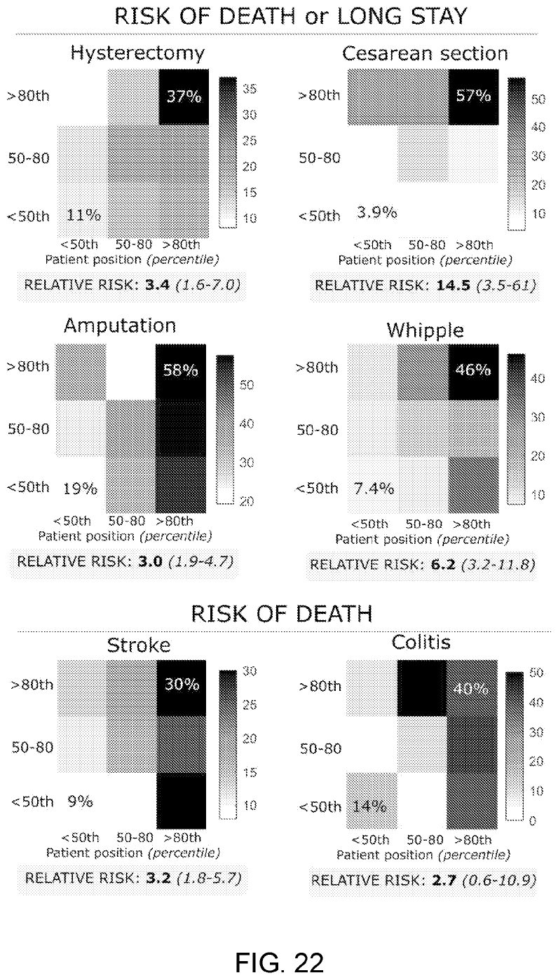

FIG. 22 shows risk stratification for other cohorts. In similar format to FIG. 5, patient risk of death or long stay, stratified by day 4 positional and directional percentiles were given for the remaining 6 inflammation cohorts. In each case, patients with position and direction above the 80^{th} percentile had significantly elevated risk comparative to patients whose position and direction were below the 50^{th} percentile. Note that due to sample size constraints, only stroke results were out-of-sample (where thresholds were defined in the exploratory cohort, and outcomes calculated from the validation cohort). For the other cohorts, thresholds and outcome rates were calculated from the overall cohort.

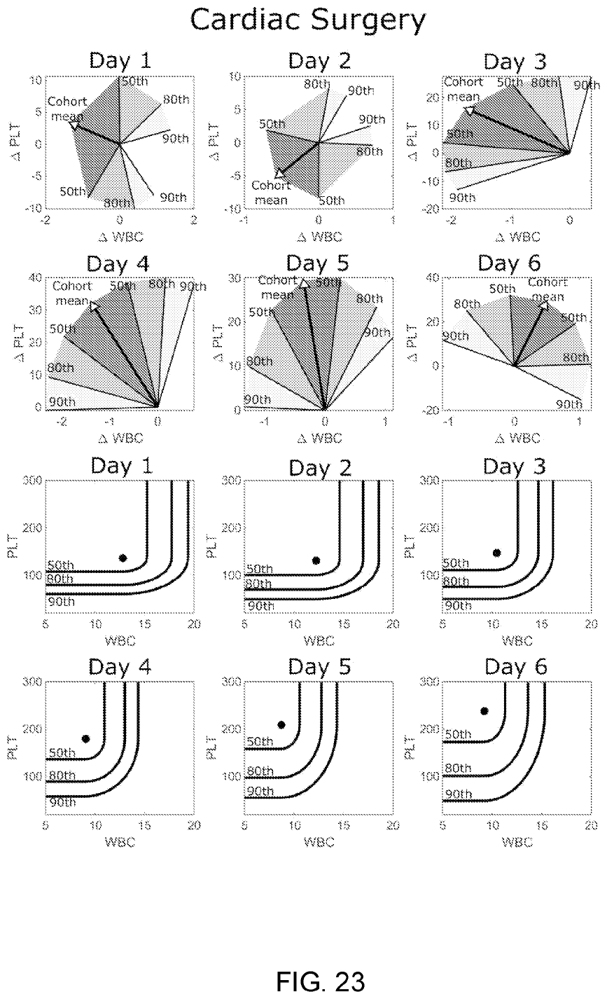

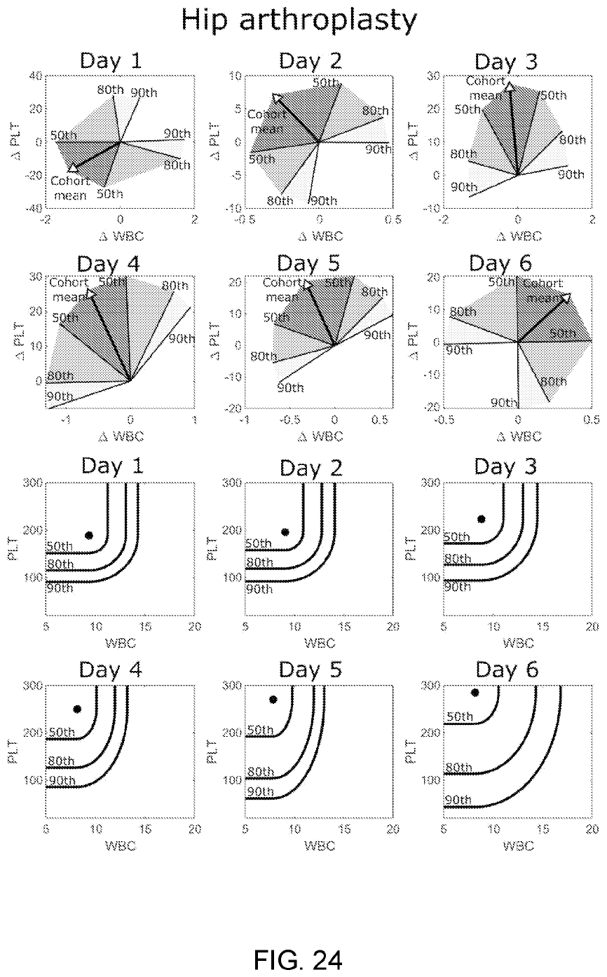

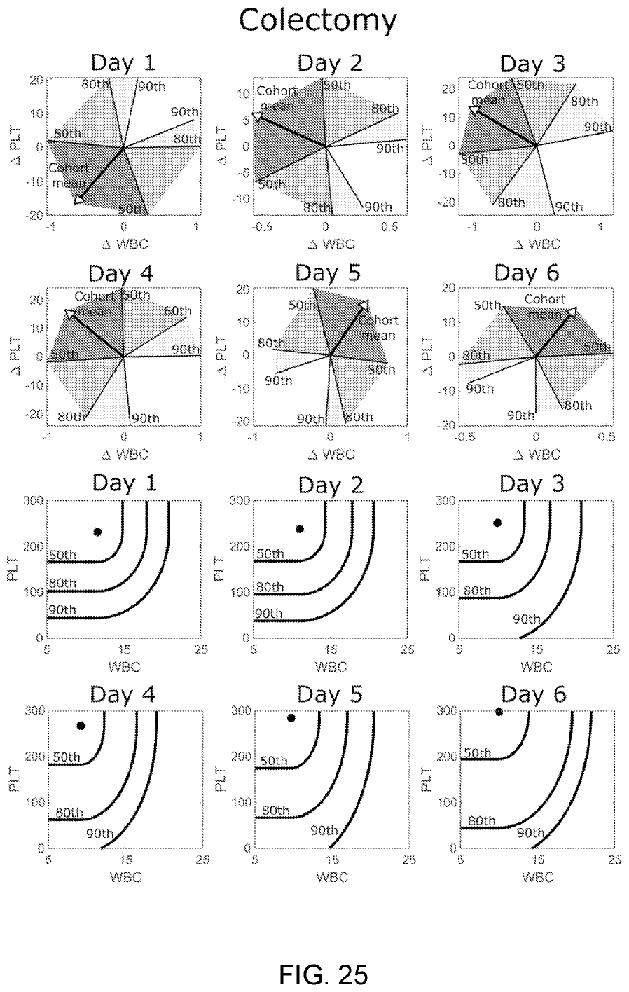

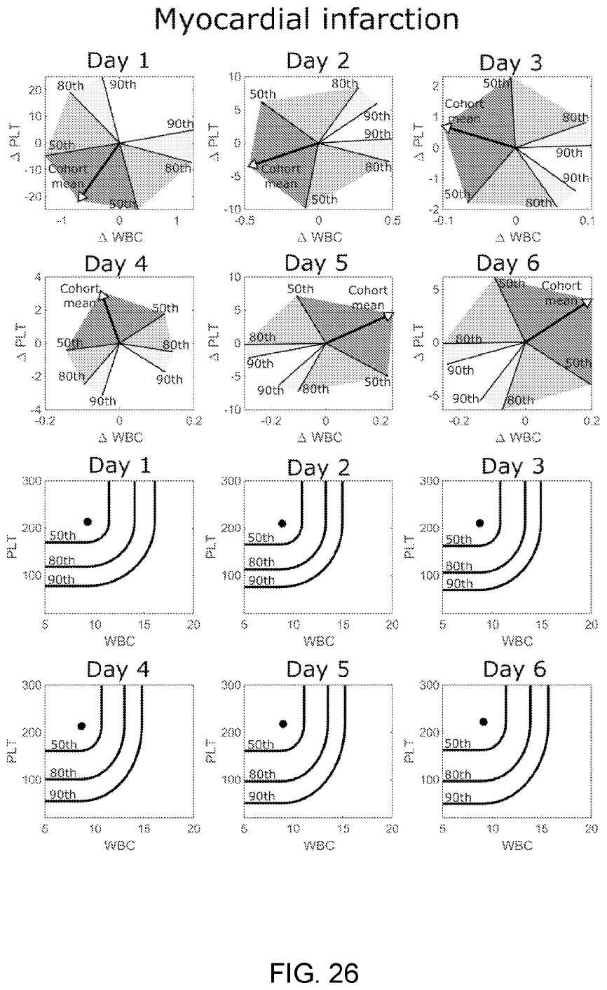

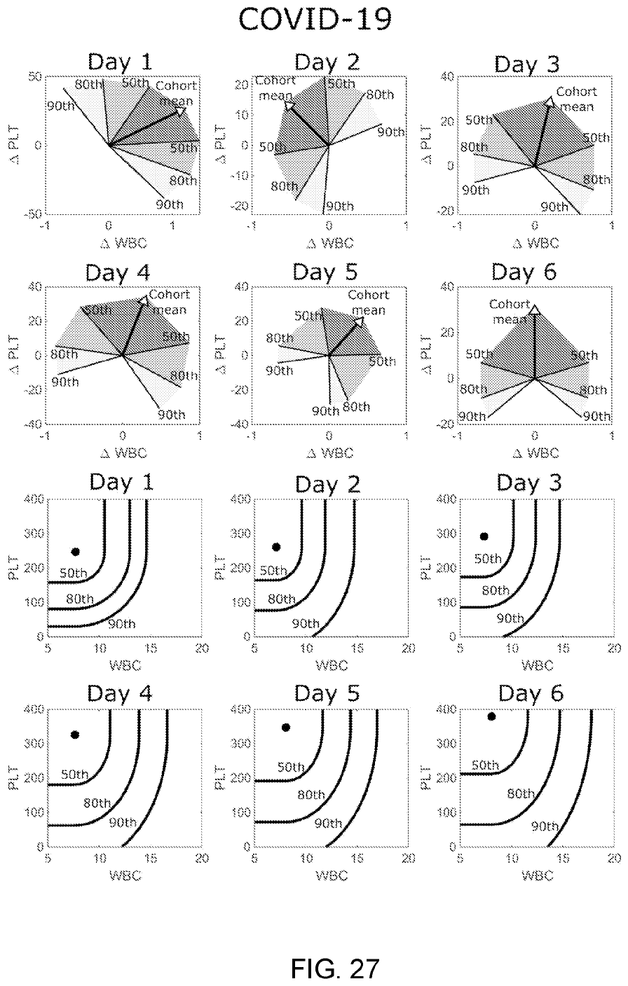

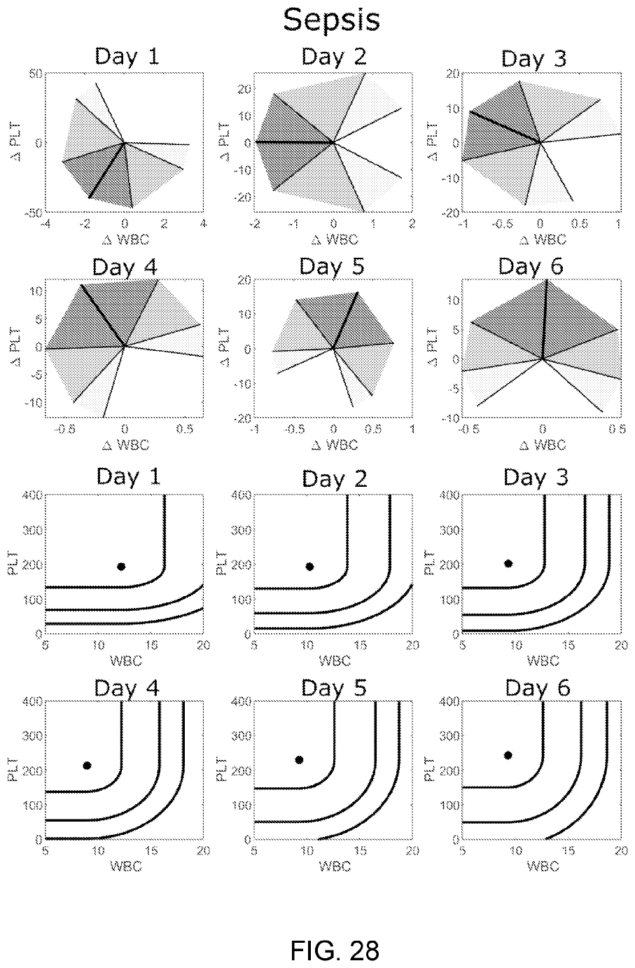

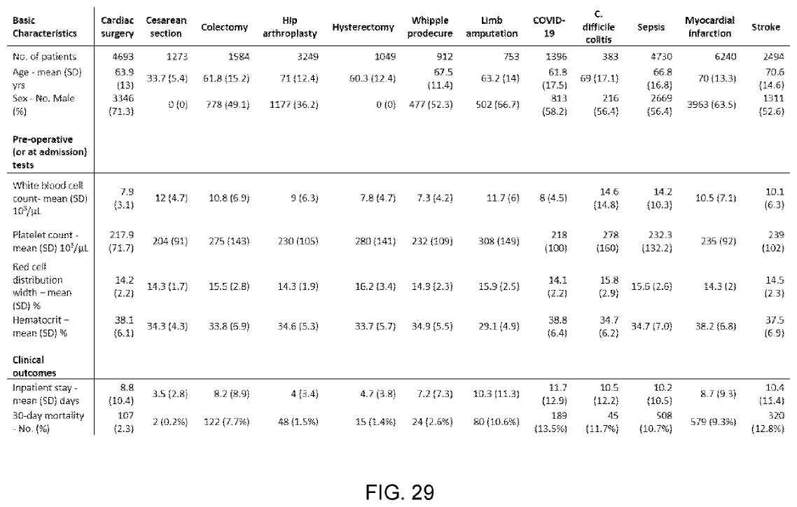

FIGS. 23-28 show position, direction, and outcome likelihoods across the first 7 days post-op for a cardiac surgery cohort (FIG. 23), a hip arthroplasty cohort (FIG. 24), a colectomy cohort (FIG. 25), a myocardial infarction cohort (FIG. 26), a COVID-19 cohort (FIG. 27), and a sepsis cohort (FIG. 28). FIG. 37 shows adverse outcomes likelihood stratified by distance and position percentiles for various inflammatory cohorts.

### REFERENCES

1. Shahian, D. M. et al. The Society of Thoracic Surgeons 2008 Cardiac Surgery Risk Models: Part 1-Coronary Artery Bypass Grafting Surgery. Ann. Thorac. Surg. (2009). doi:10.1016/j.athoracsur.2009.05.053
2. O'Brien, S. M. et al. The Society of Thoracic Surgeons 2008 Cardiac Surgery Risk Models: Part 2-Isolated Valve Surgery. Ann. Thorac. Surg. (2009). doi:10.1016/j.athoracsur.2009.05.056
3. Liu, Y., Hayes, D. N., Nobel, A. & Marron, J. S. Statistical significance of clustering for high-dimension, low-sample size data. J. Am. Stat. Assoc. (2008). doi:10.1198/016214508000000454
4. Chen, J. H. & Asch, S. M. Machine Learning and Prediction in Medicine Beyond the Peak of Inflated Expectations. N. Engl. J. Med. (2017). doi:10.1056/NEJMp1702071
5. Johnson, A. E. W. et al. Machine Learning and Decision Support in Critical Care. Proc. IEEE (2016). doi:10.1109/JPROC.2015.2501978
6. Obermeyer, Z. & Emanuel, E. J. Predicting the Future—Big Data, Machine Learning, and Clinical Medicine. N. Engl. J. Med. (2016). doi:10.1056/NEJMp1606181
7. Shameer, K., Johnson, K. W., Glicksberg, B. S., Dudley, J. T. & Sengupta, P. P. Machine learning in cardiovascular medicine: Are we there yet? Heart (2018). doi:10.1136/heartjnl-2017-311198
8. Cabitza, F., Rasoini, R. & Gensini, G. F. Unintended consequences of machine learning in medicine. JAMA—Journal of the American Medical Association (2017). doi:10.1001/jama.2017.7797
9. Strogatz, S. H. (Steven H. Nonlinear dynamics and chaos: with applications to physics, biology, chemistry, and engineering. Westview Press, a member of the Perseus Books Group (2015).
10. Medzhitov, R. Origin and physiological roles of inflammation. Nature 454, 428-435 (2008).
11. Kumar, V., Abbas, A. K., Aster, J. C., Cotran, R. S. & Robbins, S. L. (Stanley L. Robbins & Cotran pathologic basis of disease.
12. Kotas, M. E. & Medzhitov, R. Homeostasis, Inflammation, and Disease Susceptibility. Cell 160, 816-827 (2015).
13. Hall, J. E. (John E. Guyton and Hall textbook of medical physiology. (Elsevier, 2021).
14. Lin, C. C. & Segel, L. A. Mathematics applied to deterministic problems in the natural sciences. (Society for Industrial and Applied Mathematics, 1988).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
obtaining, by one or more processors, patient data, wherein the patient data includes clinical laboratory results of a patient with a disease state;
processing, by the one or more processors, the patient data such that the clinical laboratory results are normalized by patient-specific baseline levels or cohort-specific baseline levels of the clinical laboratory results; and
applying, by the one or more processors, a trajectory model to the processed patient data to identify a likelihood of an adverse outcome of the patient, in which the trajectory model characterizes the disease state by a recovery trajectory in a phase-plane of one or more variables and is configured to:
compare the processed patient data to the recovery trajectory; and
output, based on the comparing, the likelihood of the adverse outcome of the patient.

2. The method of claim 1, further comprising:
providing, by the one or more processors and based on the likelihood of adverse outcome of the patient, information indicative of a recommended treatment.

3. The method of claim 2, wherein the recommended treatment comprises a recommendation to continue a current treatment regimen or to modify the current treatment regimen.

4. (canceled)

5. The method of claim 1, wherein the clinical laboratory results are indicative of at least one of a blood count, a metabolic panel measurement, or a vital sign measurement.

6. The method of claim 1, wherein the disease state comprises an acute inflammatory response.

7.-8. (canceled)

9. The method of claim 1, wherein the one or more variables are selected from the group consistent of: a white blood cell count, a white blood cell count and a platelet count, a white blood cell count and a blood urea nitrogen level, and a white blood cell count and a red blood cell distribution width.

10.-13. (canceled)

14. The method of claim 1, wherein the trajectory model is predictive of the adverse outcome, and wherein the adverse outcome comprises at least one outcome selected from the group consisting of: a complication and mortality.

15. (canceled)

16. The method of claim 1, wherein the recovery trajectory comprises an exponential decay of white blood cell count and a linear increase in platelet count.

17. The method of claim 1, wherein comparing the processed patient data to the recovery trajectory comprises:
identifying positions of the processed patient data relative to the recovery trajectory in the phase plane; and
determining a degree to which the positions of the processed patient data deviate from the recovery trajectory at any given time point and over serial time points.

18. The method of claim 17, wherein determining the degree to which the positions of the processed patient data deviate from the recovery trajectory comprises computing a direction or angle between the positions of the processed patient data and the recovery trajectory in the phase-plane.

19. The method of claim 1, further comprising:
identifying, for the patient, the patient-specific or the cohort-specific baseline levels of the clinical laboratory results, wherein the baseline levels of the clinical laboratory results represent levels of the one or more variables before the patient is diagnosed with the disease state.

20. The method of claim 1, further comprising:
obtaining second patient data of a plurality of patients, wherein the plurality of patients does not include the patient, and the plurality of patients and the patient share patient attributes; and
imputing, based on the second patient data, missing values in the clinical laboratory results of the patient.

21.-23. (canceled)

24. The method of claim 1, wherein the trajectory model further outputs a likelihood of a full and healthy recovery of the patient.

25. The method of claim 1, further comprising:
obtaining training patient data, wherein the training patient data includes clinical laboratory results of a plurality of patients with one or more disease states;
processing the training patient data such that the clinical laboratory results of the plurality of patients are normalized by the patient-specific or cohort-specific baseline levels;
identifying the one or more variables to be used in the phase plane; and
fitting the trajectory model, using the one or more variables, to the plurality of training patient data.

26. The method of claim 25, wherein the clinical laboratory results comprise measurements of at least one parameter selected from the group consisting of: anion gap, blood-urea nitrogen, creatinine, hematocrit, glucose, platelet count, red cell distribution width, and white blood cell count.

27. The method of claim 25, wherein identifying the one or more variables comprises:
identifying, by applying unsupervised clustering to the plurality of training patient data, high-dimensional clusters, wherein the clusters are associated with the one or more disease states;
reducing dimensionality of the high-dimensional clusters; and
identifying the one or more variables that are significantly associated with the one or more disease states.

28. (canceled)

29. The method of claim 27, wherein identifying the one or more variables that are significantly associated with the one or more disease states comprises:
for a different set of the one or more variables:
computing a significance of a generalized linear model predicting the adverse outcome using the one or more variables in the plurality of training patient data; and
determining that the significance meets a threshold.

30. The method of claim 25, wherein fitting the trajectory model comprises:
fitting an exponential decay of white blood cell count using the plurality of training data; and
fitting a linear increase in platelet count using the plurality of training data.

31. A method comprising:
obtaining, by one or more processors, training patient data, wherein the training patient data includes clinical laboratory results of a plurality of patients with one or more disease states;
processing, by the one or more processors, the training patient data such that the clinical laboratory results of the plurality of patients are normalized by patient-specific or cohort-specific baseline levels;
identifying, by the one or more processors, one or more variables to be used in a phase plane; and
fitting a trajectory model, by the one or more processors and using the one or more variables, to the plurality of training patient data, in which the trajectory model characterizes the one or more disease states by a recovery trajectory in the phase plane.

32. A system comprising:
one or more processors and one or more storage devices storing instructions that are operable, when executed by the one or more processors, to cause the one or more processors to perform operations comprising:
obtaining, by one or more processors, patient data, wherein the patient data includes clinical laboratory results of a patient with a disease state;
processing, by the one or more processors, the patient data such that the clinical laboratory results are normalized by patient-specific baseline levels or cohort-specific baseline levels of the clinical laboratory results; and
applying, by the one or more processors, a trajectory model to the processed patient data to identify a likelihood of an adverse outcome of the patient, in which the trajectory model characterizes the disease state by a recovery trajectory in a phase-plane of one or more variables and is configured to:
compare the processed patient data to the recovery trajectory; and o
output, based on the comparing, the likelihood of adverse outcome of the patient.

33-35. (canceled)
